(19) United States
(12) Patent Application Publication
(10) Pub. No.: US 2015/0132778 A1
(43) Pub. Date: May 14, 2015

(54) METHODS AND KITS FOR DIAGNOSING HEPARIN-INDUCED THROMBOCYTOPENIA

(71) Applicant: The Trustees of the University of Pennsylvania, Philadelphia, PA (US)

(72) Inventors: Douglas B. Cines, Wynnewood, PA (US); Adam Cuker, Elkins Park, PA (US); Bruce Sachais, Cherry Hill, NJ (US)

(73) Assignee: The Trustees of the University of Pennsylvania, Philadelphia, PA (US)

(21) Appl. No.: 14/397,875

(22) PCT Filed: Apr. 30, 2013
(86) PCT No.: PCT/US13/38795

§ 371 Date: Oct. 30, 2014

Related U.S. Application Data
(60) Provisional application No. 61/640,960, filed on May 1, 2012.
(60) Provisional application No. 61/790,953, filed on Mar. 15, 2013.

(51) Int. Cl. G01N 33/68

(52) U.S. Cl. CPC G01N 33/6854; G01N 33/6893; G01N 2400/40; G01N 2800/222; G01N 2333/522
USPC 435/7.72

(57) ABSTRACT

Provided herein are methods for diagnosing heparin-induced thrombocytopenia (HIT) in a subject. The methods comprise (a) contacting a PF4-heparin complex with a biological sample from the subject; (b) contacting the composition of part (a) with an HIT-like antibody; and (c) measuring the amount of bound HIT-like antibody. In one embodiment, a significant decrease in the amount of bound HIT-like antibody in the composition of part (b) as compared to a control level is indicative of a diagnosis of HIT in the subject. Also provided are assay kits for performing the methods of the invention.

### STATEMENT REGARDING FEDERALLY SPONSORED RESEARCH

This invention was made with government support under grant numbers K12HL087064, R01HL078726, HL099973 and HL078726-S1 awarded by the National Institutes of Health. The government has certain rights in the invention.

### BACKGROUND OF THE INVENTION

Heparin induced thrombocytopenia (HIT) is a thrombotic complication of heparin therapy mediated by antibodies to complexes between platelet factor 4 (PF4) and heparin or glycosaminoglycans (GAGs). However, antibodies to PF4/heparin are detected by ELISA far more frequently than antibodies that activate platelets or than clinical disease. For example, anti-PF4/heparin antibodies are detected in 25-60% of patients who receive unfractionated heparin after cardiopulmonary bypass surgery and a high proportion of hospitalized patients in other medical settings, an incidence that far exceeds the prevalence of HIT.

The initial diagnosis of HIT relies on clinical impression, which may or may not receive support from subsequent laboratory evaluation. In most settings, serological assessment of HIT is confined to measurement of anti-PF4/heparin antibodies by ELISA, as more specific assays based on platelet activation are not available in real-time. HIT is uncommon even in patients receiving heparin who develop thrombocytopenia and the ELISAs most commonly employed are better suited to exclude a diagnosis of HIT than to affirm it, especially in complex medical settings where the need for a test with a high positive predictive value is most pressing. High titers of IgG antibodies correlate with platelet activation and probability of disease in experienced hands, but the outcome of predicating clinical decisions on laboratory outcomes has not been formally tested in general practice. The reason why only a fraction of patients with anti-PF4 antibodies detected by ELISA develop HIT is unclear and is only partially explained by antibody titer and IgG isotype.

One clue to the differences in the pathogenic potential of anti-PF4/heparin antibodies may begin with the finding that heparin and PF4 form complexes of diverse size that depend upon the molar ratio of the reactants. HIT antibodies and the HIT-like monoclonal antibody KKO bind and activate platelets and monocytes and promote thrombosis in an animal model over a narrow molar ratio of reactants. At these molar ratios, ultralarge complexes (ULCs) form in solution between heparin and multiple PF4 tetramers capable of binding multiple antibody molecules that in the case of platelets, may lead to sustained engagement of FcRγIIA, which initiates aggregation.

Molecular replacement studies reveal a track of amino acids on the surface of the PF4 tetramer required for binding of a HIT-like pathogenic monoclonal antibody KKO. Heparin approximates PF4 tetramers as assessed by atomic force microscopy and in doing so may expose this region or other neoepitopes recognized by pathogenic, but not by non-pathogenic antibodies, or reorganization may promote antibody avidity.

While HIT remains a major problem associated with heparin treatment, there is an absence of a robust clinical algorithm to include or to exclude its diagnosis. Furthermore the hazard associated with continuing heparin in a patient with HIT; the risk of hemorrhage in a patient with post-operative thrombocytopenia erroneously diagnosed with HIT when given a direct thrombin inhibitor; the high false positive rate of currently formulated ELISAs; and limitations in current functional assays, all converge to highlight the need for a new, rapid, reliable, portable assay to measure antibodies likely to cause disease amidst a far larger number that do not activate cells and have a low probability of being pathogenic.

### SUMMARY OF THE INVENTION

In one aspect, the invention provides a method for diagnosing heparin-induced thrombocytopenia (HIT) in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant decrease in the amount of bound HIT-like antibody in the composition of part (b) as compared to a negative control or reference level is indicative of a diagnosis of HIT in the subject.

In another aspect, the invention provides a method for diagnosing heparin-induced thrombocytopenia (HIT) in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant increase in the inhibition of binding of the HIT-like antibody in the composition of part (b) as compared to a negative control or reference level is indicative of a diagnosis of HIT in the subject.

In another aspect, the invention provides a method for ruling out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant increase in the amount of bound HIT-like antibody in the composition of part (b) as compared to a positive control or reference level indicates that the subject does not have HIT.

In another aspect, the invention provides a method for ruling out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant decrease in the inhibition of binding of the HIT-like antibody in the composition of part (b) as compared to a positive control or reference level indicates that the subject does not have HIT.

In another aspect, the invention provides a method for supporting a diagnosis of heparin-induced thrombocytopenia (HIT) in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant decrease in the amount of bound HIT-like antibody in the composition of part (b) as compared to a negative control or reference level, in combination with one or more positive clinical factors, is indicative of a diagnosis of HIT in the subject.

In another aspect, the invention provides a method for affirming a diagnosis of heparin-induced thrombocytopenia (HIT) in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant decrease in the amount of bound HIT-like antibody in the composition of part (b) as compared to a negative control or reference level, in combination with one or more positive clinical factors, is indicative of a diagnosis of HIT in the subject.

In another aspect, the invention provides a method for diagnosing an increased likelihood of heparin-induced thrombocytopenia (HIT) in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant decrease in the amount of bound HIT-like antibody in the composition of part (b) as compared to a negative control or reference level, in combination with one or more positive clinical factors, is indicative of an increased probability that the subject has HIT.

In another aspect, the invention provides a method for helping to rule out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant increase in the amount of bound HIT-like antibody in the composition of part (b) as compared to a positive control or reference level indicates that the subject is less likely to have HIT.

In another aspect, the invention provides a method for ruling out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant increase in the amount of bound HIT-like antibody in the composition of part (b) as compared to a positive control or reference level, in combination with other negative clinical factors, indicates that the subject does not have HIT.

In another aspect, the invention provides a method for helping to rule out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In this aspect, a significant increase in the amount of bound HIT-like antibody in the composition of part (b) as compared to a positive control or reference level, in combination with other negative clinical factors, indicates that the subject is less likely to have HIT.

In another aspect, the invention provides a method for diagnosing the presence of platelet-activating anti-PF4-heparin antibodies in a subject. The method includes (a) contacting a PF4-heparin complex with a biological sample from the subject; (b) contacting the composition of part (a) with an HIT-like antibody; and (c) measuring the amount of bound HIT-like antibody. In this aspect, a significant decrease in the amount of bound HIT-like antibody in the composition of part (b) as compared to a negative control or reference level indicates the presence of platelet-activating anti-PF4-heparin antibodies in the sample.

In another aspect, the invention provides a method for diagnosing the presence of platelet-activating anti-PF4-heparin antibodies in a subject. The method includes (a) contacting a PF4-heparin complex with a biological sample from the subject; (b) contacting the composition of part (a) with an HIT-like antibody; and (c) measuring the amount of bound HIT-like antibody. In this aspect, a significant increase in the inhibition of binding of the HIT-like antibody in the composition of part (b) as compared to a negative control or reference level is indicative of the presence of platelet-activating anti-PF4-heparin antibodies in the sample.

In yet another aspect, the invention provides an assay kit. The kit includes one or more of PF4 bound to heparin or a heparin-like molecule; a suitable aliquot of HIT-like antibody; a suitable aliquot of a ligand that binds HIT-like antibodies conjugated to a reporter; a suitable aliquot of a substrate which allows identification and quantification of the reporter; a solid support or bead to which the PF4-heparin can bind; and washing buffers.

Other aspects and advantages of the invention will be readily apparent from the following detailed description of the invention.

### BRIEF DESCRIPTION OF THE DRAWINGS

FIG. 1 is a line graph demonstrating the effect of pathogenic (KKO) and non-pathogenic (RTO) anti-PF4 antibodies on platelet counts in vivo. Platelet counts after intraperitoneal injection of KKO or RTO in the FcγRIIA/hPF4+ mice are shown. Platelet counts were measured before (basal=Time 0) and 4, 24, 48 and 96 hrs after antibody injection. Data are mean±1 SEM of 5 animals for each condition.

FIG. 2 is a line graph demonstrating binding of KKO and RTO measured by ELISA. PF4 alone, PF4:heparin (PF4:H), PF4^{K50E} alone, PF4^{K50E}:H. PF4^{K50E} is a mutant variant of PF4 that does not tetramerize nor form ULC of PF4-heparin and is used as a specificity control. Data are the mean±SEM of at least 3 independent experiments performed in triplicate.

FIG. 3 is a scatter plot demonstrating inhibition of KKO and RTO by human HIT antibodies. Scattergram showing inhibition of model antibody (KKO or RTO) binding to PF4:H by human plasma containing anti-PF4 antibodies (ELISA positive). “SRA Pos” denotes competition with antibodies from SRA-positive patients and “SRA Neg” denotes competition with antibodies from SRA-negative patients. Means±standard deviations are shown. *denotes p<0.0001.

FIGS. 4A and 4B demonstrate the frequency of rupture of PF4-antibody bonds in WT and PF4^{K50E} mutants. (A) The frequency of events in each 5-pN-bin was plotted against the average force for that bin after normalizing for the total number of interaction cycles. PF4 tetramers were attached covalently to pedestals and crosslinked with glutaraldehyde and Ab was covalently attached to latex beads in the absence of heparin. Each curve represents about 10,000 contact cycles of bead to pedestal. The probability of KKO binding to PF4 is much greater than for RTO, and the binding strength is slightly higher. Inset histogram shows the cumulative binding probability for KKO and RTO. (B) Similar analysis of KKO and RTO interactions with the glutaraldehyde-treated PF4^{K50E} mutant that does not form tetramers. In this case, the probability of binding of KKO is lower and comparable to that of RTO. Inset histogram shows the cumulative binding probability for KKO and RTO.

FIG. 5 is an autoradiogram of ¹²⁵I-PF4 after incubation with KKO or RTO. MM markers are to the left of each panel correspond to 55, 71, 117 and 268 kDa. Lanes 1 and 4 show PF4 complexes with (1) KKO or (4) RTO, which were subsequently crosslinked with bis-sulfosuccinimidyl suberate (BS3), a chemical crosslinker with a linking arm of 11.4 Å. Lanes 2 and 5 show PF4 crosslinked with BS3 and lanes 3 and 6 show PF4 alone (un-crosslinked). Higher molecular mass PF4 complexes in the presence of the KKO comprise ˜60% of the total PF4 tetramers vs. ˜2% with RTO. Data are representative of three such experiments.

FIG. 6 is a schematic diagram of pathogenic vs. non-pathogenic antibody binding. Simplest model showing distinction between effects of heparin on binding of pathogenic (KKO) and non-pathogenic (RTO) anti-PF4 antibodies. Heparin (orange) binds to a circumferential band of cationic residues on the surface of each PF4 tetramer (blue); the interrupted line represents binding to the distal side of the tetramer. Heparin neutralizes cationic charge repulsion among PF4 tetramers forming oligomeric complexes (shown here as a dimer for simplicity), which approximates the binding sites for KKO (yellow). Epitope approximation increases the avidity of KKO through increased proximity to more than one binding site on PF4 (1A). Some KKO antibodies may bind to epitopes on neighboring tetramers stabilizing ULCs induced by heparin (1B). In contrast, heparin has no such effect or may partially inhibit exposure of the epitope recognized by RTO (2).

FIGS. 7A-7C are scatterplots showing the OD reading of SRA+and SRA− samples in various assay formats. FIG. 7A shows data from the prior art polyspecific ELISA. FIG. 7B shows data from the IgG-specific ELISA. FIG. 7C shows data from the KKO inhibition assay of the invention. The horizontal lines represent the mean for each data set.

FIG. 8 is a line graph showing the ROC curves for each of the 3 assays described in FIGS. 7A-7C, and the AUROCs. Note that the gold standard for this analysis was an intermediate or high clinical probability of HIT (as defined by 4Ts score) and a positive SRA. HIT is defined as a 4Ts score ≧4 and +SRA. KKO inhibition cut-off of 57.63% associated with 95% sensitivity and 81% specificity.

FIGS. 9A-9D represent results from HIT-negative and HIT-positive subjects for the polyspecific ELISA, IgG-specific ELISA, KKO-I, and DT40-luc (panels A-D, respectively). Solid horizontal lines represent mean values. Dashed horizontal lines represent the cut-off associated with the most northwest point on the receiver operating characteristic curve (i.e. the cut-off at which sensitivity and specificity are optimized) for each assay.

FIG. 10 shows ROC curves for the polyspecific ELISA, IgG-specific ELISA, KKO-I, and DT40-luc. The AUCs for these assays were 0.82, 0.76, 0.93, and 0.89, respectively. The AUC for KKO-I was significantly greater than the AUC for the polyspecific (p=0.020) and IgG-specific ELISA (p=0.0044). The AUC for DT40-luc was significantly greater than the AUC for the IgG-specific (p=0.046), but not the polyspecific ELISA (p=0.28).

### DETAILED DESCRIPTION OF THE INVENTION

A robust and reproducible new assay is provided to diagnose the presence or absence of heparin-induced thrombocytopenia (HIT) in a subject. In addition, an assay is provided to determine whether platelet-activating antibodies are present or absent in a sample. The novel methods described herein derive, in part, from the inventor's discovery that a fundamental difference exists between the binding properties of pathogenic and non-pathogenic anti-PF4 antibodies.

Before describing the present invention in detail, it is intended that this invention is not limited to particular compositions or biological systems, which can, of course, vary. It is also intended that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

### I. DEFINITIONS

The terms “heparin-induced thrombocytopenia” or “HIT” and “heparin-induced thrombocytopenia and thrombosis” or “HITT” are used herein interchangeably. HIT refers to a serious, immune system—mediated complication of heparin therapy often resulting in devastating thromboembolic outcomes. HIT occurs in approximately 1% of patients exposed to therapeutic doses of unfractionated heparin for 5-10 days. HIT is a severe prothrombotic disease, with affected individuals having a 20-50% risk of developing new thromboembolic events, and has a mortality rate of about 20% with an additional about 10% of patients requiring amputations or suffering other major morbidity. Since a large number of hospitalized patients are exposed to heparin, HIT is a major iatrogenic cause of morbidity and mortality in this patient population.

The term “PF4” as used herein refers to platelet factor 4 which is a 70 amino acid, lysine-rich, 7.8 kDa platelet-specific protein that belongs to the CXC (or beta) chemokine subfamily, in which the first two of the four conserved cysteine residues are separated by one amino acid residue. The PF4 may be derived from any species that natively expresses the protein. In one embodiment, PF4 is naturally occurring, i.e., wild-type. In another embodiment, PF4 may be synthesized by recombinant or chemical methods. In another embodiment, PF4 also refers to variants or mutants thereof in which one or more of the amino acids is replaced with a different amino acid. Examples of PF4 mutations are described in International Patent Publication No. WO 02/006300, which is incorporated herein by reference. In one example, the mutated PF4 contains Glu28 and/or Lys50 mutations. In addition, fragments of PF4 including functional fragments are also encompassed by the term. In a preferred embodiment, the PF4 is human PF4.

The term “heparin” as used herein refers to a heparin or a preparation derived there from, and thus includes unfractionated heparin, low molecular weight heparin (LMWH), ultra-low molecular weight heparin (ULMWH) and the like. In addition heparin-like molecules and heparinoid molecules, are encompassed by the term. Heparin may be derived from any mammalian animal that produces the molecule. In one embodiment, the heparin is derived from a pig. In another embodiment, the heparin is derived from a cow. In another embodiment, heparin is derived from a human. In another embodiment, the heparin is synthetic. Polyanions used as a substitute for heparin are also encompassed by the term “heparin”. In one embodiment, the term heparin refers to polyvinylsulfonate.

The term “PF4-heparin complex”, as used herein, refers to any complex formed by PF4 and heparin. The term refers to both ultralarge complexes (ULCs) and small complexes (SC). The term “ULC”, as used herein, refers to ultralarge complexes of heparin and PF4, which are the most pathogenic complexes of these components. Heparin:PF4 complexes smaller than 600 kDa are typically referred to as small complexes (SC). PF4:heparin ULCs are more pathogenic than heparin:PF4 SCs. Heparin:PF4 ULCs are better recognized by HIT antibodies and lead to more platelet activation in the presence of these antibodies. In one embodiment, ULCs are 600 kDa or larger. In another embodiment, ULCs are 670 kDa or larger. In one embodiment, the PF4-heparin complex comprises a heparin substitute.

The term “antibody”, as used herein, refers to any naturally occurring or synthetic antibody or fragment thereof. Antibodies used in the methods of the invention include monoclonal antibodies, polyclonal antibodies, chimeric antibodies, antibody fragments (e.g., Fab, and F(ab′)₂) and recombinantly produced binding partners. Polyclonal antibodies may be readily generated by one of ordinary skill in the art from a variety of warm-blooded animals such as horses, cows, various fowl, rabbits, mice, or rats. Monoclonal antibodies may also be readily generated using conventional techniques.

### II. METHODS

In one aspect, the invention provides a method for diagnosing heparin-induced thrombocytopenia (HIT) in a subject. The method includes (a) contacting a PF4-heparin complex with a biological sample from the subject; (b) contacting the composition of part (a) with a HIT-like antibody; and (c) measuring the amount of bound HIT-like antibody. In one embodiment, a significant change in the amount of bound HIT-like antibody in the composition of part (b) as compared to a reference level indicates a diagnosis of HIT in the subject.

In one embodiment, the subject providing the test sample is a mammalian subject. The mammalian subject may be a human or any domestic, laboratory or farm animal. In one embodiment, the term mammalian subject includes human, mouse, pig, horse, cat, non-human primate, dog, rat, hamster, goat, bovine or ovine. In a preferred embodiment, the mammalian subject is a human.

The term “biological sample” refers to a body sample from any animal, but preferably is from a mammal, more preferably from a human. In one embodiment, the sample is any biological sample which may contain antibodies or fragments thereof. In one embodiment, the biological sample is whole blood, serum, plasma, or purified immunoglobulin. In another embodiment, the biological sample is vitreous fluid, lymph fluid, synovial fluid, follicular fluid, seminal fluid, amniotic fluid, milk, whole blood, urine, cerebro-spinal fluid, saliva, sputum, tears, perspiration, mucus, tumor lysates, and tissue culture medium, as well as tissue extracts such as homogenized tissue, tumor tissue, and cellular extracts. Such samples may further be diluted with saline, buffer or a physiologically acceptable diluent. Alternatively, such samples are concentrated by conventional means.

Although anti-PF4/heparin antibodies are detected in 25-60% of patients who receive unfractionated heparin after cardiopulmonary bypass surgery and a high proportion of hospitalized patients in other medical settings, only a small proportion of these patients develop HIT. This is due, in part, to the fact that only a portion of anti-PF4-heparin complex antibodies are pathogenic, i.e., activate platelets and lead to HIT. As used herein, the term “HIT antibody” or “platelet-activating antibody” refers to an anti-PF4-heparin complex antibody that activates platelets. The term “HIT-like antibody” refers to an antibody which behaves identically, or nearly identically, to an HIT antibody. In one embodiment, the HIT-like antibody exhibits several features which are critically similar to key features of the polyclonal human antibodies which participate in the pathogenesis of HIT. These features include preferential binding to a PF4/heparin complex relative to binding of the antibodies with either PF4 or heparin alone, specific binding of the antibody to complexes of PF4 with other sulfated glycosaminoglycans (GAGs) besides heparin, and platelet activation in the presence of the PF4/heparin complex. The HIT and HIT-like antibodies used herein may be derived from any mammalian animal. In one embodiment, the mammalian animal from which the antibody is derived is a human, mouse, pig, horse, cat, non-human primate, dog, rat, hamster, goat, bovine or ovine. In a preferred embodiment, the mammalian animal is a mouse. In another embodiment, the mammalian animal is a human. In a preferred embodiment, the HIT-like antibody is the murine monoclonal antibody designated KKO (CEDARLANE® Laboratories Limited). Certain HIT-like antibodies useful in the invention are described in U.S. Pat. No. 7,728,115, which is incorporated herein by reference. RTO is a monoclonal antibody that recognizes both PF4 and PF4-heparin and is a model for anti-PF4 antibodies that do not result in HIT, and is used herein as a model non-pathologic antibody.

The amount of HIT-like antibody bound to the PF4-heparin complex in the test well is measured and compared to a control or reference level. In one embodiment, the reference level is a pre-determined value known to correlate with a positive or negative result. As used herein, a “positive result” means a result which correlates with the presence of platelet-activating antibodies in the sample, a diagnosis of HIT or an increased risk/likelihood that the subject has HIT. As used herein, a “negative result” means a result which correlates with a lack of platelet-activating antibodies in the sample, a negative HIT diagnosis, or a decreased risk/likelihood that the subject has HIT.

In another embodiment, the reference level may be determined based on a negative or positive control assay performed prior to, after, or simultaneously with the test sample assay. As used herein, a positive control or reference level corresponds to a level of binding (or inhibition of binding as the case may be) between the PF4-heparin complex and the HIT-like antibody in the presence of platelet-activating antibodies, or a value corresponding thereto. In one embodiment, the positive control level is determined using a sample containing known platelet-activating antibodies. In another embodiment, the positive control level is determined using KKO or a HIT-like antibody or a fragment or mutant thereof. As used herein, a negative control or reference level corresponds to a level of binding (or inhibition of binding as the case may be) between the PF4-heparin complex and the HIT-like antibody in the absence of platelet-activating antibodies, or a value corresponding thereto. In one embodiment, the negative control level is determined using a sample which is known to be negative for platelet-activating antibodies. In another embodiment, the negative control level is the level of binding in the absence of a biological sample. In another embodiment, the negative control level is the level of binding in the presence of non-platelet activating anti-PF4-heparin antibodies. In another embodiment, the negative control level is the level of binding in the presence of a blank or buffer control.

In one embodiment, the level of binding between the PF4-heparin complex and the HIT-like antibody is compared to a negative control. In this embodiment, a significant decrease in the level of binding between the PF4-heparin complex and the HIT-like antibody as compared to the negative control indicates a diagnosis of HIT. As used herein, a “decrease in binding” is equivalent to an increase in inhibition binding, which means that there is less PF4-heparin:HIT-like antibody binding in the test sample than in the reference or control. In one embodiment, a “significant decrease” means a decrease in biding of 20% or more as compared to the control level (antibody binding of 80% or less of the control level). In one embodiment, a “significant decrease” means a decrease of 30% or more (antibody binding of 70% or less) as compared to the negative control level. In one embodiment, a “significant decrease” means a decrease of 35% or more as compared to the negative control level. In one embodiment, a “significant decrease” means antibody binding of 40% or more as compared to the negative control level. In one embodiment, a “significant decrease” means a decrease of 45% or more as compared to the negative control level. In one embodiment, a “significant decrease” means a decrease of 50% or more as compared to the negative control level. In one embodiment, a “significant decrease” means a decrease of 55% or more as compared to the negative control level. In one embodiment, a “significant decrease” means a decrease of 60% or more as compared to the negative control level. In one embodiment, a “significant decrease” means a decrease of 65% or more as compared to the negative control level. In one embodiment, a “significant decrease” means a decrease of 70% or more as compared to the negative control level. In one embodiment, a “significant decrease” means a decrease of 75% or more as compared to the negative control level. In another embodiment, a “significant decrease” means a decrease of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more as compared to the negative control level.

In another embodiment, a significant decrease in the level of binding between the PF4-heparin complex and the HIT-like antibody as compared to the negative control, in combination with one or more positive clinical factors, indicates a diagnosis of HIT. Positive clinical factors associated with HIT include, but are not limited to, an intermediate or high probability of HIT as defined by 4Ts score (thrombocytopenia, timing of platelet count fall, thrombosis or other sequelae, and other causes for thrombocytopenia), development of one or more thromboembolic complications (TEC), trauma/orthopedic surgery, thrombocytopenia, enlargement or extension of a previously diagnosed blood clot, development of a new blood clot, stroke, myocardial infarction, acute leg ischemia, deep vein thrombosis (DVT), pulmonary embolism (PE); systemic reaction beginning at the site of heparin infusion, including fever, chills, high blood pressure, a fast heart rate, shortness of breath, and chest pain, and rash. See, e.g., Lo G K, et al, J Thromb Haemost. 2006 April; 4(4):759-65; Crowther et al, J Crit Care. 2010 June; 25(2):287-93. Epub 2010 Feb. 10; Greinacher et al, Thromb Haemost 2005; 94: 132-5, each of which is incorporated herein by reference. Still other positive clinical factors can be determined by a clinician skilled in the art of differential diagnosis.

In another embodiment, the invention provides a method for diagnosing an increased likelihood of HIT in a subject. The method includes(a) contacting a PF4-heparin complex with a biological sample from the subject; (b) contacting the composition of part (a) with a HIT-like antibody; and (c) measuring the amount of bound HIT-like antibody. In one embodiment, a significant decrease in the amount of bound HIT-like antibody in the composition of part (b) as compared to a reference level indicates an increased likelihood that the subject has HIT. In another embodiment, a significant decrease in the amount of bound HIT-like antibody in the composition of part (b) as compared to a reference level, in combination with one or more clinical factors, indicates an increased likelihood that the subject has HIT. In another embodiment, a significant change in the amount of bound HIT-like antibody in the composition of part (b) as compared to a reference level, in combination with one or more clinical factors, indicates a diagnosis of HIT. In one embodiment, an “increased likelihood” means that the subject is more likely than a subject without a positive test result to have HIT. In another embodiment, an “increased likelihood” means that the subject is more likely than not to have HIT. In another embodiment, an “increased likelihood” means the subject has a 5, 10, 15, 20, 25, 30, 35, 40, 45, 50, 55, 60, 65, 70, 75, 80, 85, 90, 95, 100% or greater risk of having HIT than a subject without a positive test result.

In another embodiment, the level of binding between the PF4-heparin complex and the HIT-like antibody is compared to a positive control. In this embodiment, a lack of a significant change in binding of PF4-heparin complex and the HIT-like antibody in the test sample as compared to the positive control indicates a positive diagnosis of HIT.

In another aspect, the invention provides a method for ruling out a diagnosis of HIT in a subject. The method includes (a) contacting a PF4-heparin complex with a biological sample from the subject; (b) contacting the composition resulting from part (a) with an HIT-like antibody; and (c) measuring the amount of bound HIT-like antibody. In one embodiment, a significant increase in the amount of bound HIT-like antibody in the composition of part (b) as compared to a positive control or reference level indicates that the subject does not have HIT. As used herein, an “increase in binding” is equivalent to a decrease in inhibition of binding, which means that there is more PF4-heparin:HIT-like antibody binding in the test sample than in the reference or positive control. In one embodiment, a “significant increase” means an increase in antibody binding of 10% or more as compared to the positive control level. In one embodiment, a “significant increase” means an increase in antibody binding of 15% or more as compared to the positive control level. In one embodiment, a “significant increase” means an increase in antibody binding of 20% or more as compared to the positive control level. In one embodiment, a “significant increase” means an increase in antibody binding of 25% or more as compared to the positive control level. In one embodiment, a “significant increase” means an increase in antibody binding of 30% or more as compared to the positive control level. In one embodiment, a “significant increase” means an increase in antibody binding of 35% or more as compared to the positive control level. In one embodiment, a “significant increase” means an increase in antibody binding of 40% or more as compared to the positive control level. In one embodiment, a “significant increase” means an increase in antibody binding of 45% or more as compared to the positive control level. In one embodiment, a “significant increase” means an increase in antibody binding of 50% or more as compared to the positive control level. In one embodiment, a “significant increase” means an increase in antibody binding of 55% or more as compared to the positive control level. In one embodiment, a “significant increase” means an increase in antibody binding of 60% or more as compared to the positive control level. In one embodiment, a “significant increase” means an increase in antibody binding of 65% or more as compared to the positive control level. In one embodiment, a “significant increase” means an increase in antibody binding of 70% or more as compared to the positive control level. In one embodiment, a “significant increase” means an increase in antibody binding of 75% or more as compared to the positive control level. In one embodiment, a “significant increase” means an increase in antibody binding of 80% or more as compared to the positive control level. In one embodiment, a “significant increase” means an increase in antibody binding of 85% or more as compared to the positive control level. In one embodiment, a “significant increase” means an increase in antibody binding of 90% or more as compared to the positive control level. In one embodiment, a “significant increase” means an increase in antibody binding of 95% or more as compared to the positive control level. In one embodiment, a “significant increase” means an increase in antibody binding of 100% or more as compared to the positive control level. In another embodiment, a “significant increase” means an increase of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 6, 65, 66, 67, 68, 69, 70, 71, 72, 72, 74, 75, 76, 77, 78, 79, 80, 81, 82, 83, 84, 85, 86, 87, 88, 89, 90, 91, 92, 93, 94, 95, 96, 97, 98, 99, 100% or more.

In another embodiment, a significant increase in the amount of bound HIT-like antibody in the composition of part (b) as compared to a positive control or reference level, in combination with other negative clinical factors, indicates that the subject does not have HIT. Negative clinical factors associated with a decreased risk of HIT include a low probability of HIT as defined by 4Ts score and lack of one or more of the positive clinical factors described above. Still other negative clinical factors can be determined by a clinician skilled in the art of differential diagnosis.

In another embodiment, the invention provides a method for helping to rule out a diagnosis of HIT in a subject. The method includes (a) contacting a PF4-heparin complex with a biological sample from the subject; (b) contacting the composition of part (a) with a HIT-like antibody; and (c) measuring the amount of bound HIT-like antibody. In one embodiment, a significant increase in the amount of bound HIT-like antibody in the composition of part (b) as compared to a positive control or reference level indicates a decreased likelihood that the subject has HIT. In another embodiment, a significant increase in the amount of bound HIT-like antibody in the composition of part (b) as compared to a positive control or reference level, in combination with one or more clinical factors, indicates a decreased likelihood that the subject has HIT. In another embodiment, a significant increase in the amount of bound HIT-like antibody in the composition of part (b) as compared to a positive control or reference level, in combination with one or more clinical factors, indicates that the patient does not have HIT. In one embodiment, a “decreased likelihood” means that the subject is less likely than a subject with a positive test result to have HIT. In another embodiment, a “decreased likelihood” means that the subject is more likely than not to be free of HIT. In another embodiment, a “decreased likelihood” means the subject has a 95, 90, 85, 80, 75, 70, 65, 60, 55, 50, 45, 40, 35, 30, 25, 20, 15, 10, 5 or less risk of having HIT than a subject with a positive test result.

In one embodiment, a diagnosis of HIT in the subject or the presence of platelet-activating antibodies in the sample is made based on the percent inhibition of KKO binding. In one embodiment, “0%” represents no inhibition of KKO binding and “100%” represents complete inhibition of KKO binding. In one embodiment, a test result of about 50% inhibition or greater indicates a positive test result or a diagnosis of HIT in the subject or presence of platelet-activating antibodies in the sample. In one embodiment, a test result of about 55% inhibition or greater indicates a positive test result or a diagnosis of HIT in the subject or presence of platelet-activating antibodies in the sample. In one embodiment, a test result of about 58% inhibition or greater indicates a positive test result or a diagnosis of HIT in the subject or presence of platelet-activating antibodies in the sample. In one embodiment, a test result of about 60% inhibition or greater indicates a positive test result or a diagnosis of HIT in the subject or presence of platelet-activating antibodies in the sample. In one embodiment, a test result of about 65% inhibition or greater indicates a positive test result or a diagnosis of HIT in the subject or presence of platelet-activating antibodies in the sample. In one embodiment, a test result of about 66% inhibition or greater indicates a positive test result or a diagnosis of HIT in the subject or presence of platelet-activating antibodies in the sample. In one embodiment, a test result of about 70% inhibition or greater indicates a positive test result or a diagnosis of HIT in the subject or presence of platelet-activating antibodies in the sample. In one embodiment, a test result of about 75% inhibition or greater indicates a positive test result or a diagnosis of HIT in the subject or presence of platelet-activating antibodies in the sample. In one embodiment, a test result of about 80% inhibition or greater indicates a positive test result or a diagnosis of HIT in the subject or presence of platelet-activating antibodies in the sample. In one embodiment, a test result of about 85% or greater inhibition indicates a positive test result or a diagnosis of HIT in the subject or presence of platelet-activating antibodies in the sample.

In some embodiments, the amount of bound HIT-like antibody in the sample is compared to both a positive and negative control. In certain embodiments, an increase in binding as compared to the positive control and a decrease in binding as compared to the negative control indicate a positive diagnosis of HIT in the subject. As a non-limiting example, when the positive control has 10% binding (90% inhibition) and the negative control has 95% binding (5% inhibition), a test result of 40% binding (60% inhibition) would indicate a diagnosis of HIT in the subject or presence of platelet-activating antibodies in the sample.

In another embodiment, the level of binding between the PF4-heparin complex and the HIT-like antibody is compared to a negative control. In this embodiment, a lack of a significant change in binding of PF4-heparin complex and the HIT-like antibody in the test sample as compared to the negative control indicates a negative diagnosis of HIT.

In another aspect, the invention provides a method for diagnosing the presence of platelet-activating anti-PF4-heparin antibodies in the sample. The method includes (a) contacting a PF4-heparin complex with a biological sample from the subject; (b) contacting the composition of part (a) with an HIT-like antibody; and (c) measuring the amount of bound HIT-like antibody. In one embodiment, a significant decrease in the amount of bound HIT-like antibody in the composition of part (b) as compared to a negative control or reference level indicates the presence of platelet-activating anti-PF4-heparin antibodies in the sample.

In another embodiment, the level of binding between the PF4-heparin complex and the HIT-like antibody is compared to a positive control. In this embodiment, a lack of a significant change in binding of PF4-heparin complex and the HIT-like antibody in the test sample as compared to the positive control indicates the presence of platelet-activating anti-PF4-heparin antibodies in the sample.

In certain embodiments, the method further comprises performing one or more additional diagnostic tests to confirm the diagnosis of HIT or the presence of platelet-activating antibodies in the sample. In one embodiment, the additional diagnostic tests are one or more of a polyspecific ELISA (e.g., available from HYPHEN BioMed™), an IgG-specific ELISA (e.g., TECHNOZYM® assay available from Technoclone), a cell based assay and a serotonin release assay (SRA), which are known in the art. See, e.g., U.S. Provisional Patent Application No. 61/614,729, which is incorporated herein by reference, for description of a cell based assay useful in the invention. See, e.g., MOREL-KOPP, M.-C., ABOUD, M., TAN, C. W., KULATHILAKE, C. and WARD, C. (2011), Heparin-induced thrombocytopenia: evaluation of IgG and IgGAM ELISA assays. International Journal of Laboratory Hematology, 33: 245-250 which is incorporated by reference herein.

### III. ASSAY FORMATS

The assays as described herein may be adapted to many of the assay formats which are well known and conventional in the art. In certain embodiments, the assay employed utilizes the PF4-heparin complex bound to a surface. In one embodiment, the assay is in the form of an enzyme-linked immunosorbent assay (ELISA) that utilizes PF4-heparin complexes as capture reagents for platelet-activating and HIT-like antibodies. Techniques for preparing plates for and performing ELISAs are well known in the art. See, e.g., Crowther, J R, ELISA: Theory and Practice, Vol. 42, 1^{st} Ed., Springer-Verlag, 2010, and Arepally et al., 1995. Am. J. Clin. Pathol., 104:648 which are incorporated by reference herein. Many different variants of ELISA methods exist. The description below aims at illustrating a typical ELISA technique. It does not presume to be complete and should not be construed in any way as restricting the scope of the present invention. For example, in the following description, ELISA methods are described as comprising separate steps of incubating a biological sample with the PF4-heparin complex and HIT-like antibody and incubating the reaction product formed with a secondary antibody. However, some existing ELISA embodiments do not comprise such separate incubation steps and allow the PF4-heparin complex to react simultaneously, or shortly one after the other, in one and the same incubation step, with both the HIT-like antibody and secondary antibody. The subject invention is in principle applicable to any and all ELISA variants, and to similar immunoassay methods which, strictly speaking, are not ELISA methods, e.g., because they do not involve the use of an enzyme.

In certain embodiments, the PF4-heparin complex is used in an immobilized form, i.e. attached to a solid phase, such as polystyrene beads or the inner surface of the reaction container (e.g. a reaction tube or a well of a microtiter plate). The PF4-heparin complex may be physically adsorbed onto the solid phase or, usually, be attached by covalent binding. In some ELISA embodiments, the PF4-heparin complex is attached by using a suitable coupling agent, and in others by using appropriate linker substances, such as biotin and (strept)avidin.

In one embodiment, the solid surface is coated with PF4 in the presence of heparin. The appropriate relative concentrations of PF4 and heparin can be readily determined by one of skill in the art, e.g., using stoichiometric calculations and previously published estimates of specific activity of 140 U/mg and a mean MW of 15 kDa. The amount of heparin relative to PF4 is critical as the assay uses heparin and PF4 in molar ratios that optimize the formation of ULC. In one embodiment, the molar ratio is about near 1:1 given that PF4 is a tetramer and the average molecular mass of heparin is 15 kDa. In another embodiment, PF4 is added at a concentration of 1 ug/mL to 20 ug/mL. In a preferred embodiment, the PF4 is present at 5 ug/mL. In one embodiment, the heparin is added at a concentration of 0.1 U/mL. In another embodiment, heparin is added at a concentration of 0.05 U/mL. In another embodiment, heparin is added at a concentration of 0.2 U/mL. ELISA plates having PF4-heparin (or a heparin-like molecule or substitute as described above) are available commercially (GTI® Diagnostics, HYPHEN Biomed™, STAGO™) and may be used in certain embodiments of the invention.

In another embodiment, a non-reacting protein, such as bovine serum albumin or casein, is added to block any plastic surface in the well that remains uncoated by the PF4-heparin complex. In one embodiment, the plates are washed one or more times after the PF4-heparin binding step and/or after the blocking step. The washing medium may be any suitable buffer known in the art. In one embodiment, the buffer is phosphate buffered saline (PBS) with or without additional components, such as detergents, preferably Tween. In another embodiment, the buffer is tris-buffered saline, with or without additional components.

The biological sample suspected of containing platelet-inducing anti-PF4-heparin antibodies is contacted with the PF4-heparin complex and allowed to incubate. In all steps, incubation can occur under normal human physiological conditions (i.e., 37° C.), room temperature, or other conditions as can be determined by one skilled in the art. The appropriate incubation time can be determined by one of skill in the art based on the concentration of reagents used, the expected concentration of platelet-activating antibodies in the sample, etc. In one embodiment, the assay plate is allowed to incubate for 1 minute to 1 hour. In a preferred embodiment, the assay plate is allowed to incubate for 30 minutes.

The biological sample may be any of those described above, including whole blood, plasma, serum or purified immunoglobulin. The sample may be concentrated or diluted using conventional means. In one embodiment, the sample is diluted 1:50 with buffer. The sample is added at a volume appropriate for the reaction vessel. If platelet-activating anti-PF4-heparin antibodies are present in the sample, they will bind the PF4-heparin complex.

In some ELISA embodiments, the immobilization of the PF4-heparin complex is carried out after the incubation of the biological sample and PF4-heparin complex, thereby allowing the reaction between the PF4-heparin complex and the antibody to proceed in the liquid phase. To allow its subsequent immobilization, the PF4-heparin complex may be applied in a biotinylated form Immobilization can then be effected by using a solid phase carrying (strept)avidin.

A HIT-like antibody is then added to the test mixture and incubated to allow binding with the PF4-heparin complex. The appropriate concentration of HIT-like antibody may be determined by one of skill in the art. In one embodiment, the concentration of HIT-like antibody is about 1 ng/mL to about 1 mg/mL. In a preferred embodiment, the concentration of HIT-like antibody is about 0.01 μg/mL. In another preferred embodiment, the concentration of HIT-like antibody is about 0.02 μg/mL. In a preferred embodiment, the concentration of HIT-like antibody is about 0.05 μg/mL. In a preferred embodiment, the test mixture is allowed to incubate for 5 minutes at 37° C. The plates are washed one or more times after the HIT-like antibody is incubated.

The amount of bound HIT-like antibody is then measured. In some embodiments, this is accomplished using a ligand that binds the HIT-like antibody. In a preferred embodiment, the ligand is a secondary antibody which recognizes the HIT-like antibody. In one embodiment, the ligand is an IgG antibody. In certain embodiments, the HIT-like antibody is modified in such a way as to allow its detection, as discussed below so that the addition of a ligand that binds the antibody is not necessary. In one embodiment, the HIT-like antibody is conjugated to biotin or an enzyme.

In certain embodiments, the ligand carries a label/reporter molecule allowing its detection. In some ELISA embodiments, however, the ligand is used in unlabeled form and is labeled after its binding by using a labeled binding partner for the ligand. As an example thereof, the ligand may be a mouse antibody (either polyclonal or monoclonal) against the HIT-like antibody, and after its binding to the PF4-heparin complex, a labeled goat anti-mouse IgG is used to attach a label to the immobilized complex.

As used herein, a detectable “label” includes any molecule which may be detected directly or indirectly so as to reveal the presence of the PF4-heparin-HIT-like antibody complex in the sample. Many direct and indirect labels are known in the art and are useful in the invention. See, e.g., US Patent Publication No. 2011/0177500, which is incorporated herein by reference. In some embodiments of the invention, a direct detectable label is used. In certain embodiments the label is an enzyme, a fluorochrome, a luminescent or chemi-luminescent material, or a radioactive material. In one embodiment, the label consists of an enzyme capable of a detectable conversion of a substrate, e.g. a peroxidase such as horseradish peroxidase, capable of converting, in the presence of hydrogen peroxide, a substrate, such as 3,3′5,5′-tetramethylbenzidine (TMB), into a colored product. In a preferred embodiment, the reporter molecule is an enzyme capable of being detected by color change when contacted with a chromogenic substrate. In one embodiment, the method comprises contacting the composition with the chromogenic substrate and detecting the color change via spectrophotometer. In one embodiment, the reporter molecule is horseradish peroxidase or alkaline phosphatase. In another embodiment, the HIT-like antibody is associated with, or conjugated to a fluorescent detectable fluorochrome. Commonly used fluorochromes include fluorescein isothiocyanate (FITC), phycoerythrin (PE), allophycocyanin (APC), coriphosphine-O (CPO) and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

In some embodiments, after the labeled ligand has been attached to the HIT-like antibody, the solid phase with PF4-heparin:HIT-like antibody complex bound thereto is washed before the actual detection phase is entered. In certain embodiments, in the detection phase, substrate solution is added to the solid phase with attached complex and the conversion, if any, of the substrate is detected. The substrate may be any known in the art. As a non-limiting example, the substrate is selected from p-Nitrophenyl Phosphate (PNPP), 2,2′-Azinobis[3-ethylbenzothiazoline-6-sulfonic acid (ABTS), o-phenylenediamine dihydrochloride (OPD), and 3,3′,5,5′-tetramethylbenzidine (TMB).

To allow quantitative measurement of the analyte, the solid phase is incubated with the substrate solution for a fixed time, which should be sufficiently long to allow a substantial enzymatic conversion of the substrate into a colored substance, and may be determined by one of skill in the art. In one embodiment, the fixed time is between 1 minute and 5 hours. In another embodiment, the fixed time is between 5 minutes and 1 hour. In another embodiment, the fixed time is 20-30 minutes. After termination of the substrate-converting reaction the intensity of the coloration, which is proportional to the immobilized amount of enzyme, is measured by optical means, such as a spectrophotometer to measure the absorbance at a chosen wavelength, such as 405 nm, 450 nm or 490 nm. In one embodiment, the substrate-converting reaction is terminated by the addition of a stop solution.

In other embodiments, a Western blotting assay is used in which the PF4-heparin complex is the antigen (See, Harlow et al., 1988, Antibodies: A Laboratory Manual, N.Y., Cold Spring Harbor Laboratory, 479-504). In another embodiment, a flow cytometry assay is used to assess the level of the bound HIT-like antibody in which microspheres are employed. In one embodiment, the microspheres are used in solution and not bound to a solid support. Such assay systems include the xMAP® system from Luminex Corp®. Briefly, fluorescent labeled microspheres are coupled to the PF4-heparin complex and incubated with the biological sample and the HIT-like antibody in solution. The amount of bound HIT-like antibody is detected using magnetic technology in which the microspheres with the bound complex are detected using a flow cytometer instrument or CCD camera.

Other methods known in the art for assessing the level or concentration of a bound HIT-like antibody in the test sample can be used, such as, by way of example and not by limitation, chromatographic methods or other immunological methods (see, for example, Harlow et al., 1988, Antibodies: A Laboratory Manual, Cold Spring Harbor, N.Y., which is incorporated herein by reference). For example, particle-gel immunoassay methods can be used which employ a particle gel immunoassay commonly employed in transfusion medicine. Briefly, red high density polystyrene beads coated with human PF4/heparin complexes are exposed to HIT-like antibodies and a biological sample in a reaction chamber containing a buffered sephacryl gel matrix. The beads are centrifuged and agglutination at the top of the dispersed gel is interpreted as positive detection. See, e.g, Pouplard et al, J Thromb Haemost. 2007 July; 5(7):1373-9. Epub 2007 Mar. 14, which is incorporated by reference herein. Enzyme immunoassay (EIA) methods can also be used. An EIA is a fluid phase assay which measures, e.g., the binding of HIT-like antibodies to biotinylated-PF4/heparin complexes in solution (See, e.g. Newman et al., 1998, Thrombosis and Haemostasis. 80:292-297) which is incorporated herein by reference.

In yet another embodiment of the method of the invention, the measuring is performed by a computer processor or computer-programmed instrument that generates numerical or graphical data useful in diagnosing the presence of HIT or platelet-activating antibodies.

### IV. KITS

In another aspect, assay kits are provided. In one embodiment, the assay kit includes one or more of PF4 bound to heparin or a heparin-like molecule; a suitable aliquot of HIT-like antibody; a suitable aliquot of a ligand that binds HIT-like antibodies conjugated to a reporter; a suitable aliquot of a substrate which allows identification and quantification of the reporter; a solid support or bead to which the PF4-heparin can bind; and washing buffers. In one embodiment, the assay kit is used to perform any of the methods described above. For conciseness, each and every embodiment of each component is not repeated here. However, it is intended that each of the embodiments of the assay components shall have the same scope as the embodiments described above.

Suitably, the kit contains packaging or a container with each of the above-described components. In one embodiment, the kit contains instructions on how to perform the assay and optionally, additional materials for performing such assays including, e.g., reagents, well plates, containers, markers or labels, and the like.

The compositions of these kits also may be provided in dried or lyophilized forms. When reagents or components are provided as a dried form, reconstitution generally is by the addition of a suitable solvent. It is envisioned that the solvent also may be provided in another packaging means.

The kits of the present invention also will typically include a means for containing the vials in close confinement for commercial sale such as, e.g., injection or blow-molded plastic containers into which the desired vials are retained. Other instrumentation includes devices that permit the reading or monitoring of reactions in vitro.

### V. EXAMPLES

The examples that follow do not limit the scope of the embodiments described herein. One skilled in the art will appreciate that modifications can be made in the following examples which are intended to be encompassed by the spirit and scope of the invention.

To begin to understand the structural basis of pathogenic antibodies, we compared the effect of heparin on the binding of KKO and platelet activating anti-PF4/heparin antibodies and anti-PF4/heparin antibodies not associated with platelet activation from patients suspected of HIT. The data presented here indicate there is a fundamental difference between the binding properties of pathogenic and non-pathogenic anti-PF4 antibodies that is not evident by ELISA.

### Example 1

### Reagents

A. Generation of human PF4 in Schnieder 2 (S2) insect cells. cDNAs encoding human WT PF4 and PF4^{K50E} were cloned into the plasmid pMT/BiPN5-His (Invitrogen Corp., Carlsbad Calif.) for expression in Drosophila Expression System (Invitrogen). Cloning was performed using Bgl II and Age I cloning sites. A hexanucleotide encoding Bgl II site was then eliminated by site-directed mutagenesis so that the expressed protein contained full-length wild type (WT) PF4 or PF4^{K50E} with an identical sequence as their counterparts expressed in E. coli. PF4 expression was induced by adding copper sulfate (0.5 mM) and the protein was collected in serum-free medium Insect-Xpress (Lonza, Walkersville, Md.) for 3-5 days; sodium azide (0.02% final concentration) and EDTA (2.5 mM final concentration) were added, and the media was filtered through Express Plus 0.22 mm filter (Millipore Corp., Billerica, Mass.). PF4 was purified from the media on a heparin HiTrap column (GE Healthcare) on an ATKA Prime FPLC (GE Healthcare) at 4° C. using a 10 mM Tris, 1 mM EDTA, pH 8 buffer system. Medium was loaded in buffer containing 0.5 M NaCl and PF4 eluted at 1.8 M NaCl using a linear gradient. Fractions containing purified PF4 detected by silver staining of 12% polyacrylamide gels (SDS-PAGE) were pooled, concentrated and buffer exchanged into 50 mM HEPES, 0.5 M NaCl, pH˜7.2 using an Amicon Ultra filter (3000 molecular weight cut-off, Millipore). Protein was quantified using a BCA assay (Pierce). PF4^{K50E} was purified as WT PF4 above with several modifications: the buffer system used was 50 mM MES, 1 mM EDTA, pH 6.5; media was loaded in buffer containing 0.3 M NaCl, and the protein was eluted at 1.3 M NaCl using a linear gradient.

B. Anti-human PF4 monoclonal antibodies to PF4. KKO and RTO hybridoma cells were generated and characterized as previously described (Arepally et al, Blood, 2000, 95(5):1290-5 which is incorporated herein by reference). Briefly, KKO and RTO are IgG2bK monoclonal anti-human PF4 antibodies generated concurrently in mice injected with complexes of human PF4 and UFH at an equimolar ratio. The IgG fractions were purified from conditioned PFHM-II media (Invitrogen) using protein A agarose (Invitrogen) as recommended by the manufacturer. IgG purity was demonstrated by SDS-PAGE on NuPAGE 4-12% Bis-Tris Gel (Invitrogen).

### Example 2

### Effect of KKO and RTO on Platelet Counts in Vivo

FcγRIIA/hPF4⁺ mice, generated and characterized as previously described, were injected intraperitoneally with KKO (10 or 20 mg/kg) or RTO (10 or 100 mg/kg) and the platelet count was measured daily over the next four days. All studies involving animal were approved by the institutional animal care committee at the Children's Hospital of Philadelphia.

### Example 3

### Binding of KKO and RTO to PF4: ELISA

Immulon 4 HBX plates (Thermo Electron Corp., Milford Mass.) were coated overnight at room temperature (RT) with PF4 or PF4^{K50E} (50 μl/well, 5 μg/ml) in PBS, in the absence or presence of 0.1 U/ml heparin (Hospira Inc, Lake Forest, Ill.). For stoichiometric calculations, we utilized previously published estimates of specific activity of 140 U/mg and a mean MW of 15 kDa. The plates were washed 4 times with 180 μl PBS, and non-reactive sites were blocked with 1% BSA in PBS (150 μl/well) for 1 hr at RT. Serial dilutions of KKO or RTO IgGs were added in 1% BSA/PBS (100 μl/well) for 1 hr at RT. The plates were washed 6 times with 180 μl PBS/0.1% Tween-20. HRP-conjugated goat anti-mouse IgG-Fc (Bethyl Laboratories, Inc., Montgomery Tex., Cat. No. A90-131P) diluted 1:3,000 in 1% BSA/PBS was added (100 μl/well) for 1 hr at RT and washed 6 times with 180 μl PBS/0.1% Tween-20. HRP substrate ABTS (Roche, Mannheim, Germany, Ref 11 204 521 001) dissolved in ABTS buffer as recommended by the manufacturer was added (100 μl/well) at RT. Color development over time was read on a SpectraMax 340 (Molecular Devices, Sunnyvale Calif.) at 405 nm and 490 nm.

### Example 4

### Inhibition of KKO and RTO Binding: ELISA

Plates were coated with PF4:heparin and blocked with 1% BSA as above. Human plasma (1:50 dilution) was added in 1% BSA/PBS (50 μl/well) for 30 min at 37° C., followed by KKO or RTO (0.02 μg/mL) ±plasma (50 μl/well) for 5 min at 37° C. Plates were washed 5 times with 180 μl PBS/0.1% Tween-20. HRP-conjugated goat anti-mouse IgG-Fc (Jackson Laboratories, West Grove, Pa.: Prod #115-035-207) diluted 1:5,000 in 1% BSA/PBS was added (50 μl/well) for 30 min at 37° C. and washed 5 times with 180 μl PBS/0.1% Tween-20. The assay was performed and color development over time was measured as above.

### Example 5

### Binding of Human HIT Antibodies to PF4: ELISA

Citrated plasma samples from 15 patients referred to the Coagulation Laboratory at the Hospital of University of Pennsylvania for evaluation of HIT were selected for analysis based on serologic profile. All patients experienced a >30% fall in platelet count in appropriate temporal relationship to heparin exposure to raise concern for HIT. Samples from eight patients produced a positive polyspecific ELISA result (Gen-Probe GTI Diagnostics, Waukesha, Wis.) and a positive in-house serotonin release assay result (SRA); 7 patients had a positive ELISA and a negative SRA. The mean optical density (OD) result in both groups was >1.0 OD unit (strong positive). ELISAs were performed as described above using wells precoated with PF4±heparin. Plasma was tested at 1:50 dilution. The investigator performing these assays was blinded to clinical history and serologic profile. Studies were performed in accordance with approval of the Institutional Review Board at the University of Pennsylvania.

### Example 6

### Oligomerization of PF4 Assessed by Autoradiography

Radiolabeling of PF4 was performed with Na¹²⁵I (Perkin Elmer Life Sciences, Shelton, Conn.) with immobilized chloramine T (Iodo-Beads; Pierce) according to the manufacturer's instructions. Equimolar solutions of ¹²⁵I-labeled PF4¹⁶ (10 μg/mL) and KKO or RTO (46.5 μg/mL) were incubated for 30 min at RT in a volume of 30 μL PBS. The cross-linker bis-sulfosuccinimidyl suberate (BS3; Thermo Fisher Scientific, Rockford, Ill.; final concentration 0.2 mM) or PBS was added for an additional 30 min at room temperature. Oligomerization of PF4 was examined by SDS-PAGE (3-8% tris-acetate gradient gel). The relative amounts of PF4 migrating as tetramers or at higher molecular masses was quantified by densitometry using the Gel Logic 100 imaging system with Kodak molecular imaging software (V4.5.1).

### Example 7

### Binding of KKO and RTO to PF4: Optical Trap-Based Force Spectroscopy

To measure the binding of KKO and RTO to PF4 at the single-molecule level while minimizing effects of avidity and other auxiliary intermolecular interactions, we used optical trap-based force spectroscopy that we developed (not shown). In optical trap-based force spectroscopy, the tension produced on the receptor-attached ligand-coated latex bead causes a beam deflection that is sensed by a photodetector and displayed as a voltage signal, reflecting the strength of ligand-receptor binding (data not shown). Because of the stochastic nature and variability, rupture forces following contact are displayed as force histograms. In these experiments, a KKO- or RTO-coated 2 nm-latex bead was trapped by the focused laser light and brought to a distance of a few microns from a PF4-coated 5 μm-spherical silica pedestal (data not shown). After oscillating the bead at 10 Hz with a 0.8 μm peak-to-peak amplitude (2,000 pN/s loading rate), the bead was brought into intermittent contact with the pedestal by micromanipulation using a keyboard-controlled piezoelectric stage (data not shown). Rupture force signals following repeated contacts between the pedestal and the bead were collected for periods of up to 1 min and were displayed as normalized force histograms for each experimental condition.

To ensure the comparability of the binding probability determined for KKO vs RTO with PF4, we used identical coating protocols for both antibodies, including the same initial concentration in the binding mixture with the same freshly activated beads under identical experimental conditions. From many previous experiments with other proteins, we know that the immobilization protocol is robust and highly reproducible in terms of surface density and reactivity of proteins. To maximize single-molecule interactions while decreasing the likelihood of multiple interactions, the surface densities of reacting proteins were deliberately decreased so that the fraction of specific interactions between antibody and PF4 was approximately 10% of bead-pedestal contacts or less (See Supplement for details). Because only a small percentage of contact/detachment cycles resulted in effective antigen-antibody binding/unbinding, data from at least 10 experiments, representing 10³ to 10⁴ individual measurements were combined. Individual forces measured during each contact-detachment cycle were collected into 5 pN-wide force ranges (bins). The number of events in each bin was plotted against the average force for that bin after normalizing for the total number of interaction cycles. The percentage of events in a particular bin represents the probability density of rupture events at that tension. Importantly, to minimize potential weaker signals due to non-covalent PF4-PF4 interactions (revealed in control experiments as a noisy background), the surface-bound WT PF4 tetramers were covalently cross-linked with 0.5% glutaraldehyde prior to interaction with an antibody-coated bead. Glutaraldehyde was chosen for these initial studies because it generated homogeneous, irreversible complexes (as opposed to heparin) that preserved binding of both mouse monoclonal antibodies and human antibodies to PF4 (data not shown). The efficacy of cross-linking was confirmed as described above using SDS-PAGE.

### Example 8

### Identification of Pathogenic and Non-Pathogenic Antibody

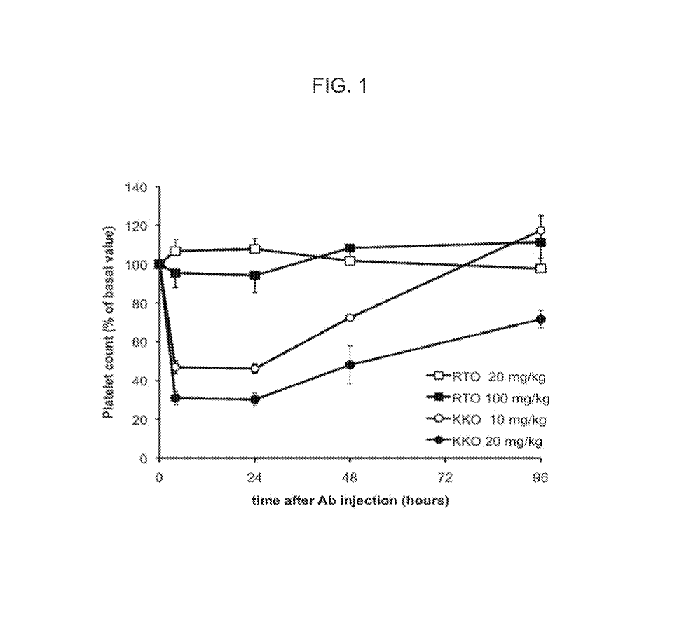

Our goal was to begin to understand the molecular basis of the difference between anti-PF4 antibodies associated with platelet activation, a central feature of HIT, and those that are not. To do so, we took advantage of two extensively characterized murine monoclonal anti-human PF4 IgG2bK antibodies, designated KKO and RTO. Consistent with our previous work, injection of KKO into FcγRIIA/hPF4⁺-expressing transgenic mice caused a transient reduction in the platelet count by approximately 70%, whereas the same amount of RTO had no effect (FIG. 1). To take into account unknown pharmacokinetic interactions that could affect antibody availability, a 10-fold higher dose of RTO was injected. Again, RTO did not cause a significant fall in the platelet count.

### Example 9

### Comparison of KKO and RTO Binding by ELISA

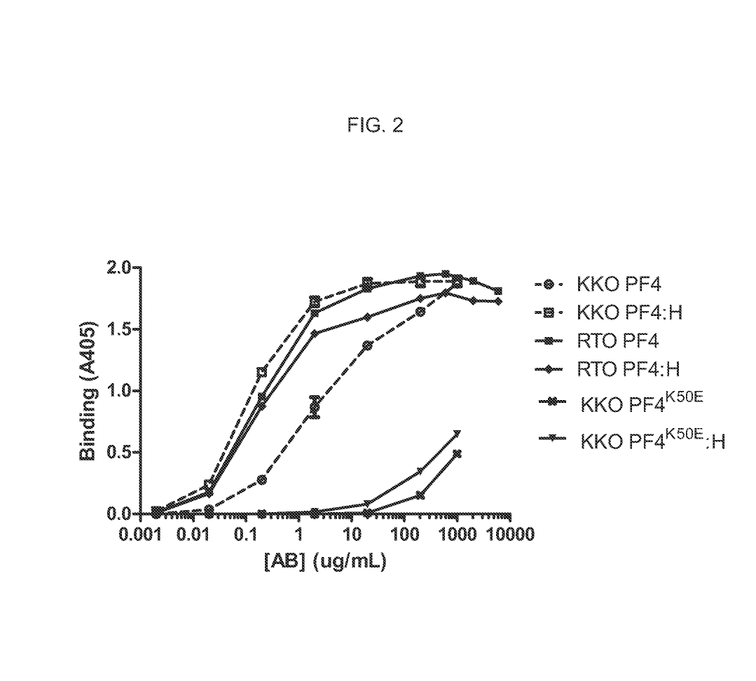

We next measured the binding properties of these two antibodies to PF4 and PF4 complexed to heparin (PF4:H) by ELISA (FIG. 2). While KKO and RTO completely inhibited binding of their biotinylated counterparts, neither inhibited the binding of the other (data not shown) suggesting they recognize distinct binding sites. RTO bound more tightly to PF4 than KKO in the absence of heparin (EC₅₀ is 0.19±0.05 μg/mL and 6.13±0.01 μg/mL, respectively). Binding of RTO was unaffected by heparin (EC₅₀=0.19±0.04). In contrast, binding of KKO to PF4:H was 32-fold higher than to PF4 alone (EC₅₀ is 0.19±0.01 and 6.13±0.01 μg/mL respectively) and comparable to binding of RTO to PF4:H. Of note, maximal binding of both antibodies to PF4 was virtually identical in the presence of heparin under conditions where secondary antibody and substrate were not limiting.

### Example 10

### Relationship Between Binding of Monoclonal and Human Anti-PF4 Antibodies

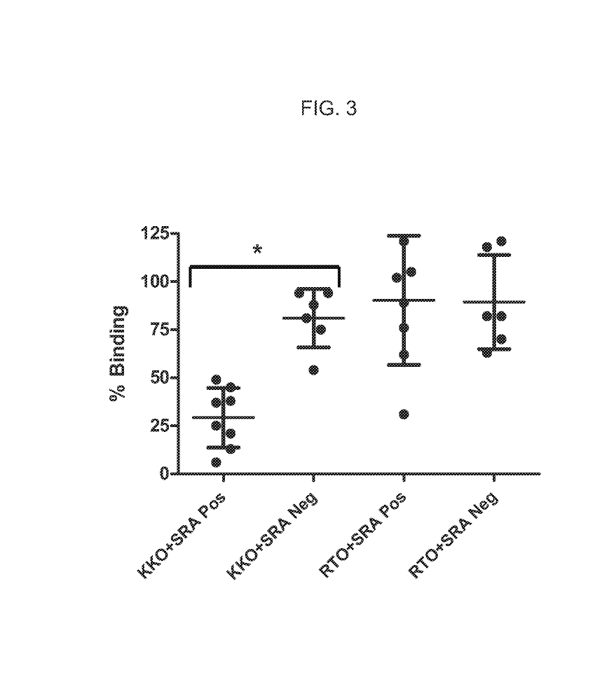

Based on these results, we asked whether anti-PF4 antibodies that activate platelets (positive SRA) from patients suspected of HIT compete for KKO binding to PF4:heparin, whereas anti-PF4 antibodies that fail to activate platelets do not. Plasma from 8 ELISA+/SRA+ patients suspected of HIT inhibited KKO binding by a mean of 80% compared with 30% by ELISA+/SRA− samples (p=<0.0001), whereas no discrimination was evident based on inhibition of RTO binding (90% vs 89%, p=0.96; FIG. 3). These data are consistent with the concept that platelet activating antibodies from patients suspected of having HIT recognize an epitope that overlaps with KKO but is less prevalent in plasma from patients without platelet activating antibodies.

### Example 11

### Dynamic Bimolecular Interactions between Surface-Bound Antibody and PF4

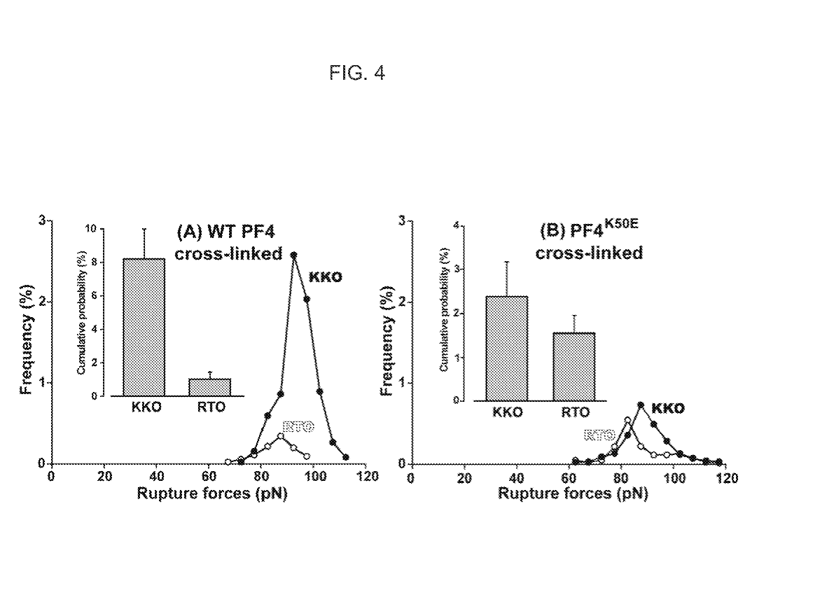

We next tested the hypothesis that the discordance between measurements of antibody binding by ELISA and their effect in vivo is due to the complications of avidity and other intermolecular interactions that are intrinsic to bulk equilibrium assays such as ELISAs. To explore the possibility that KKO and RTO bind PF4 differently at the single molecule level, we measured the binding interactions of these antibodies with PF4 using an original biophysical methodology named optical trap-based force spectroscopy (data not shown). This approach enabled us to measure the probability and strength of binding among bimolecular partners in a non-equilibrium state over a wide range of rupture forces capable of dissociating non-covalent single-molecule protein-protein complexes. Through a series of control experiments with tetrameric WT PF4 and either KKO or RTO, we identified a set of lower binding strength interactions arising from PF4-PF4 bonds (not shown), which partially overlapped with antibody-PF4 interactions. To study antibody-PF4 binding specifically, we prevented rupture of PF4-PF4 bonds by covalently cross-linking the PF4 tetramers with glutaraldehyde. The specificity of rupture forces generated by the surface-bound cross-linked PF4 and surface attached KKO or RTO was confirmed by competitive inhibition experiments in the presence of free antibodies or PF4 (not shown). The force histogram revealed that KKO-PF4 interactions occurred with about an 8-fold higher probability than RTO-PF4 interactions at the same surface densities, reflecting a much higher reactivity of KKO to PF4 (FIG. 4A, inset). KKO-PF4 interactions were also slightly stronger, as reflected by the position of the peak at higher rupture force (FIG. 4A). In contrast to the WT PF4, KKO and RTO showed lower and similar binding probabilities to glutaraldehyde-treated PF4^{K50E}, which forms few tetramers or higher-ordered complexes (FIG. 4B).

### Example 12

### Role of PF4 Oligomerization in Antibody Binding

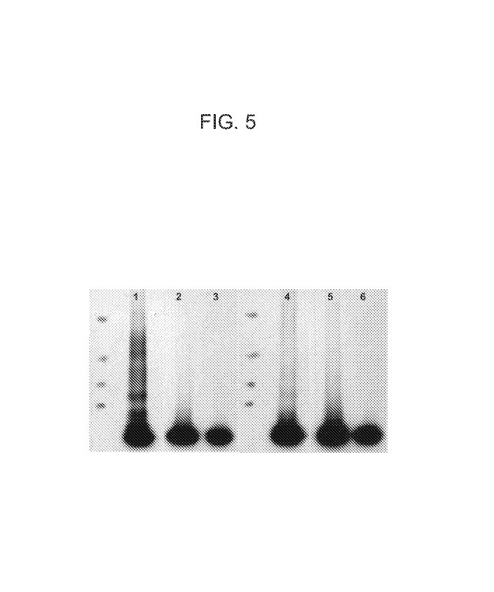

In view of these differences, we next examined the impact of these two antibodies on oligomerization of PF4, which is induced by heparin. PF4^{K50E} has a single amino acid mutation at the interface between dimer surfaces. This sharply curtails tetramer formation, which we have shown is a prerequisite for heparin-induced oligomerization and binding of HIT antibodies. Binding of both KKO and RTO to PF4^{K50E} was weak and insensitive to heparin, confirming the importance of PF4 tetramerization (FIG. 4B). We then hypothesized that KKO, like heparin, may approximate PF4 tetramers, increasing its avidity in the ELISA and in vivo, whereas RTO does not. To explore this possibility, we examined whether KKO and/or RTO clustered PF4. Incubation of PF4 with KKO, but not RTO, generated larger molecular mass structures (FIG. 5). As both antibodies bind PF4 in the presence of heparin with almost identical EC₅₀'s and maximal capacity by ELISA, this suggests that KKO, unlike RTO, promotes PF4 clustering of complexes (120-200 kDa) like heparin, though to a lesser extent (>700 kDa). As the linking arm of BS3 used in these experiments is only 11.4 Å and crosslinks primary amines on the side-chains of lysine residues and amino termini, these data may demonstrate only a subset of such complexes that are formed.

### Example 13

### Discussion

The finding that the prevalence of anti-PF4/heparin antibodies vastly exceeds clinical disease in settings such as cardiopulmonary bypass surgery, among others, raises the question of why only a subset of antibodies detected by ELISA is associated with the risk of developing thrombocytopenia and thrombosis. We hypothesized that part of the explanation involves heterogeneity in the distribution of PF4 bound to heparin or heparin-like compounds in ELISA wells as compared to ultra-large complexes (ULCs) formed in solution or on cell surfaces. It is likely that ULC formation in the ELISA plate may be partly constrained (by steric and/or electrostatic effects), limiting the formation (or accessibility) of critical binding epitopes needed to optimize binding of HIT antibodies. In addition, the ELISA format yields a measurement of total antibody binding at equilibrium, which contrasts with the situation in vivo where the opportunity for antibody-antigen interactions with cell surface GAGs are likely brief and subject to hemodilution and disruption by flow.

The binding was compared of two isotype matched monoclonal IgG anti-human PF4 antibodies: KKO, which causes thrombocytopenia (and thrombosis) in vivo, and RTO, which does not. The epitopes recognized by the antibodies differed based on cross-competition experiments and greater inhibition of KKO by SRA-positive but not SRA-negative human plasma, in contrast to RTO for which no specific pattern of inhibition by human antibody was seen. Importantly both antibodies bound comparably to PF4/heparin-coated wells as measured using a standard ELISA employed clinically, but adapted to measure mouse antibody, eliminating the question of titer or isotype from consideration.

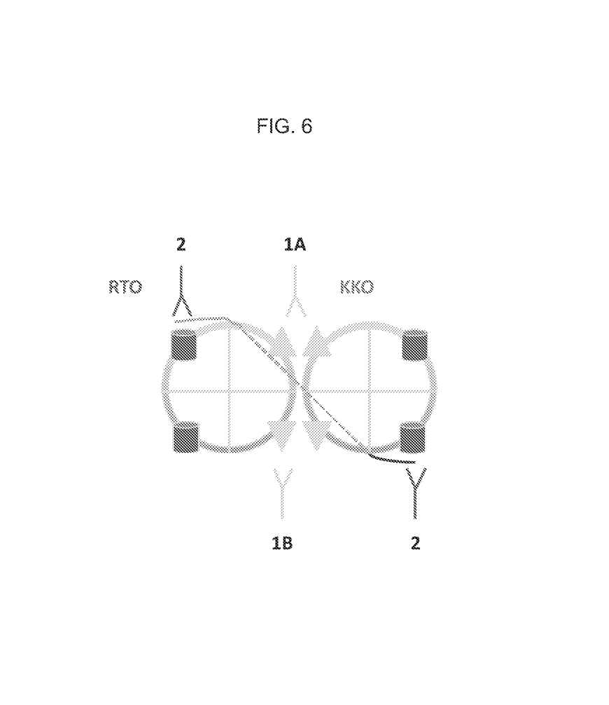

These two antibodies differed in several important characteristics. KKO induced oligomerization of PF4, as does heparin, whereas RTO did not, and heparin enhanced the avidity of KKO, but had no effect on RTO. We hypothesized that the binding avidity of KKO, and possibly HIT antibodies with which it competes, is enhanced when heparin or cellular GAGs promote organization of PF4 tetramers into higher-ordered complexes in vitro or in vivo. These complexes are in turn stabilized by KKO (FIG. 6). In contrast, high titers of non-pathogenic anti-PF4 antibodies may show comparable behavior at equilibrium, but their binding is not enhanced by heparin-induced oligomerization and they bind to sites that do not reinforce oligomer stability (FIG. 6).

In furtherance of this concept, measurement of antibody-PF4 binding at the single-molecule level (using rupture force spectroscopy) rather than in bulk (by ELISA) reveals that the probability of KKO binding to PF4 was approximately 8-fold greater than RTO binding and that higher tensile forces, such as may occur with shear in vivo, were required to dissociate KKO. This increase was most apparent when PF4 was oligomerized and was not seen when a non-oligomerizing PF4 variant, recognized by both antibodies, was studied. Although studying PF4-antibody interactions at the single-molecule level is somewhat artificial in terms of surface density, steric limitations due to surface confinement and relative orientation, and may or may not reflect antibody behavior in vivo, this approach provides important information on the fundamental nature of these interactions that correlates with the in vivo activity of these antibodies which was not apparent by ELISA. In two dimensional kinetics, the binding probability at a given contact duration and surface densities of interacting molecules reflects the rate of association and is governed by the on-rate constant, while the peak of rupture forces (binding strength) depends on the height of the dissociation energy barrier and reflects the forced dissociation rate. Therefore, the data indicate that at least under two dimensional non-equilibrium conditions, KKO has higher affinity for PF4 tetramers than RTO irrespective of avidity and other effects of heparin- or antibody-induced, higher-order polymerization of PF4. In combination with the ability of KKO to promote “super-oligomerization” of the PF4 tetramers, these data suggest an amplification reaction, in which KKO, unlike RTO, binds preferentially to PF4 tetramers (with or without heparin), further enhances their polymerization and binding becomes more avid as the antigenic complexes grow in size (FIG. 6). Whatever mechanisms are operative, we hypothesize that the single-molecule rupture force measurements, which summarize thousands of ongoing individual antigen-antibody interactions, simulate more closely the transient interactions that vascular and hematopoietic cells are subjected to in the circulation due to hemodilution and shear forces, which might affect antibody binding in ways not reflected in the ELISA platform.

Our studies affirm and extend those previously described using atomic force microscopy by showing the importance of epitope specificity, probability and strength of antibody binding in real-time and the differences in the effect of approximation of epitopes by heparin on the binding of platelet activating vs. non-activating antibodies. Together, these results begin to reveal potentially important biological differences in the behavior of anti-PF4 antibodies not reflected in prior art ELISAs or similar in vitro detection systems. They suggest that epitope specificity may be an important parameter in assessing clinical risk, in part because oligomerization of the antigen by heparin or GAGs affects antibody binding and its downstream consequences. In turn, cell-activating antibodies may stabilize oligomer formation.

### Example 14

### Further Studies

Further experiments were performed to compare the specificity and sensitivity of the novel ELISA with assays previously used for detecting HIT or PF4 antibodies. For the “Inhibition of KKO binding” assay, Immulon 4 HBX plates (Thermo Electron Corp., Milford Mass.) were coated overnight at room temperature (RT) with PF4 (50 μl/well, 5 μg/ml) in PBS, in the presence of 0.1 U/ml heparin (Hospira Inc, Lake Forest, Ill.). The plates were washed 4 times with 180 μl PBS, and non-reactive sites were blocked with 1% BSA in PBS (150 μl/well) for 1 hr at RT. Human plasma (1:50 dilution) was added in 1% BSA/PBS (50 μl/well) for 30 min at 37° C., followed by KKO (0.02 μg/mL) ±plasma (50 μl/well) for 5 min at 37° C. KKO IgG was added in 1% BSA/PBS (100 μl/well) for 1 hr at RT. Plates were washed 5 times with 180 μl PBS/0.1% Tween-20. HRP-conjugated goat anti-mouse IgG-Fc (Jackson Laboratories, West Grove, Pa.: Prod #115-035-207) diluted 1:5,000 in 1% BSA/PBS was added (50 μl/well) for 30 min at 37° C. and washed 5 times with 180 μl PBS/0.1% Tween-20. HRP substrate ABTS (Roche, Mannheim, Germany, Ref 11 204 521 001) dissolved in ABTS buffer as recommended by the manufacturer was added (100 μl/well) at RT. Color development over time was read on a SpectraMax 340 (Molecular Devices, Sunnyvale Calif.) at 405 nm and 490 nm. Polyspecific and IgG-specific ELISAs were performed according to the manufacturers protocols (GTI® Diagnostics).

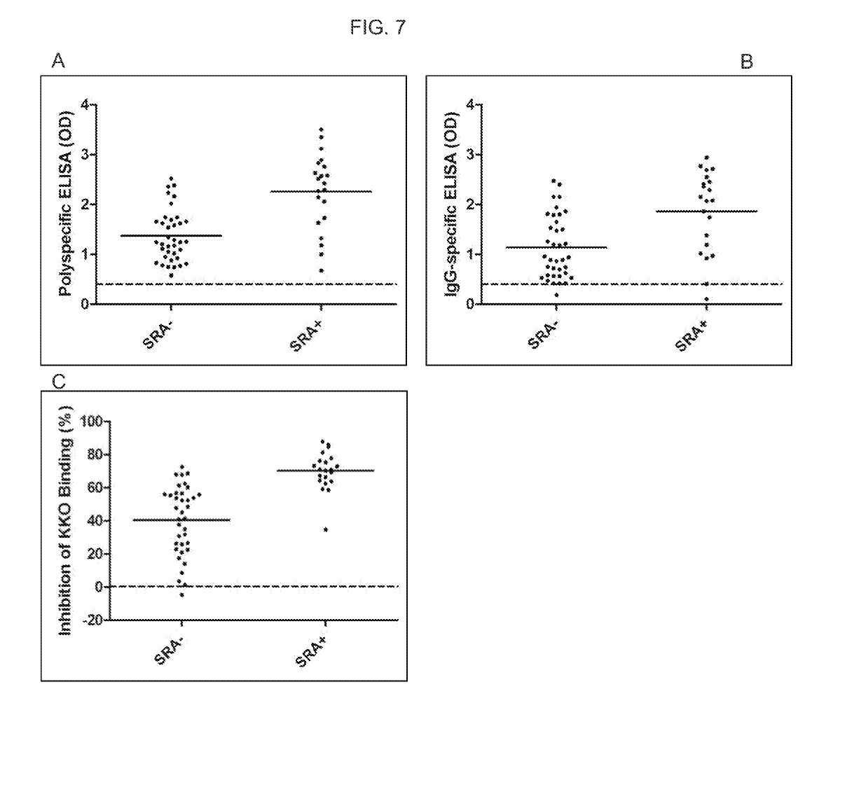

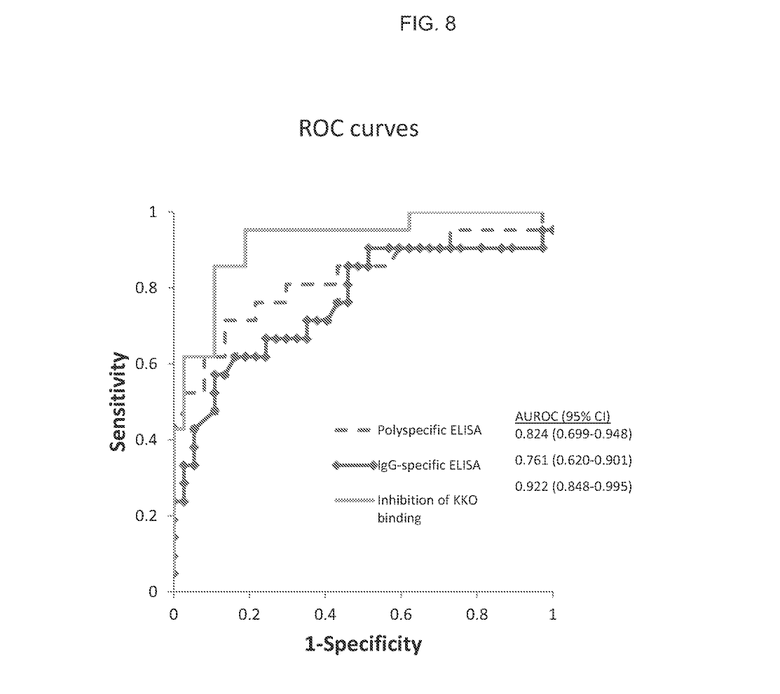

FIG. 7 shows the optical density of SRA− and SRA+ samples in each of the KKO inhibition, polyspecific ELISA and IgG-specific ELISA assays. The data show that the KKO inhibition assay discriminates better between SRA− and SRA+ individuals than the 2 commercially available ELISAs (polyspecific and IgG-specific). FIG. 8 shows the ROC curves for each of the 3 assays and the AUROCs. Note that the gold standard for this analysis was an intermediate or high clinical probability of HIT (as defined by 4Ts score) and a positive SRA. KKO inhibition cut off of 57.63% is associated with 95% sensitivity and 81% specificity.

### Example 15

### Further Comparison with Other Methods of Detecting HIT

The reference numbers in the remaining examples refer to the second set of references appended hereto.

The performance of the KKO-I assay was compared with a DT40-luc assay described in U.S. Provisional Patent Application No. 61/614,729 which is incorporated herein by reference, and two commercially available immunoassays in samples from 58 patients with suspected HIT and circulating anti-PF4/heparin antibodies.

Patient samples consecutively referred to the University of Pennsylvania for laboratory assessment of HIT that tested positive [i.e. optical density (OD)≧0.40] in a polyspecific PF4/heparin ELISA (Hologic Gen-Probe, San Diego, Calif.) were included. Citrated plasma samples from all patients were also tested using an IgG-specific PF4/heparin ELISA (Hologic Gen-Probe), an in-house SRA, and the investigational KKO-I and DT40-luc assays. The polyspecific and IgG-specific ELISAs were performed in accordance with the manufacturer's instructions. The SRA was performed with platelet rich plasma (PRP) as previously described (hereafter referred to as PRP-SRA) and was considered positive if there was <5% 14C serotonin release after patient plasma was added to platelets in the absence of heparin and >20% release after addition of 0.1 or 0.5 U/ml of heparin.

The KKO-I assay was performed as described above and as described in Sachais et al, Blood, 2012; 120(5):1137-42, which is incorporated by reference herein. Briefly, Immulon 4 HBx 96-well plates (Thermo Fisher Scientific, Waltham, Mass.) coated with PF4 and heparin (Sagent Pharmaceuticals, Schaumburg, Ill.) were incubated with human plasma (1:50 dilution) for 30 minutes at 37° C. followed by incubation with KKO for an additional 10 minutes at 37° C. Recombinant PF4 was expressed in Drosophila Schneider 2 cells and purified as previously described. KKO binding was measured as absorbance at 405 nm (A405) after incubation with HRP-conjugated goat anti-mouse IgG-Fc (Jackson ImmunoResearch Laboratories, West Grove, Pa.) and the HRP substrate ABTS (Roche Applied Science, Penzberg, Germany). Absorbance was measured with a SpectraCount plate reader (Packard BioScience, Waltham, Mass.). Data are presented as % inhibition of KKO binding, calculated as [(A405ₘₐₓ−A405ₚₐₜᵢₑₙₜ)/A405ₘₐₓ]×100 with A405ₘₐₓ set in the absence of plasma. “0%” represents no inhibition of KKO binding and 100% represents complete inhibition.

The DT40-luc assay was performed as described in Sachais et al, Blood, 2012; 199(25):5955-5962, which is incorporated by reference herein. In brief, DT40 chicken B cells were transiently transfected to express human FcγRIIA (pEF6-FcγRIIA) as well as a reporter molecule (NFATLuc), which consists of the luciferase gene under control of the IL-2 promoter. On the day following transfection, the resultant DT40-luc cells were placed in 96-well culture plates. PF4/heparin complexes were formed first by incubating recombinant PF4 with heparin for 15 minutes at 37° C., followed by addition of patient plasma (1:800 final dilution). The PF4/heparin/plasma mixtures were then added to the cells for 6 hours at 37° C. in an atmosphere containing 5% CO2. Plates were frozen at −80° C. to terminate the activation reaction. To measure activation, cells were thawed and lysed with 5× Passive Lysis Buffer (Promega, Madison, Wis.) for 15 min. Luciferase activity was measured on a Berthold (Pforzheim, Germany) MultiLumat LB 9506 Luminometer (10 sec readings) using Luciferase Assay Reagent (Promega) following the manufacturer's instructions. Data are reported as the ratio of luciferase signal induced by patient plasma relative to the absence of plasma (fold-basal). Normalization of luciferase signal in the absence of plasma was performed with each transfection to account for potential differences in transfection efficiency.

We determined the 4Ts score for each subject by retrospective chart review to estimate the clinical likelihood of HIT at the time of laboratory testing. The investigator performing 4Ts scoring (AC) was blinded to the results of all HIT laboratory assays. Investigators performing the KKO-I and DT40-luc assays (AHR, JLH) were blinded to the 4Ts score and the results of the PRP-SRA and the polyspecific and IgG-specific PF4/heparin ELISA. The protocol was approved by the University of Pennsylvania institutional review board.

HIT was defined as the combination of an intermediate or high probability 4Ts score≧4 and a positive PRP-SRA. The performance of the polyspecific ELISA, IgG-specific ELISA, KKO-I, and DT40-luc assay were evaluated with respect to this reference standard by receiver-operating characteristic (ROC) analysis. Areas under the ROC curves (AUCs) were calculated and compared by the DeLong method for correlated samples. Analyses were carried out using GraphPad Prism 5 (GraphPad Software, La Jolla, Calif.) and Analyse-it (Analyse-it Software, Leeds, UK). A p-value<0.05 was considered statistically significant.

### Example 16

### Results

Plasma samples from 58 subjects were studied. Each of the 21 subjects tested positive by PRP-SRA had a 4Ts score≧4 and thus met the prespecified criteria for HIT. Plasma samples from 37 subjects tested negative by PRP-SRA and were considered negative for HIT. Demographic and clinical characteristics of HIT-positive and HIT-negative subjects are summarized in Table 1. The median 4Ts score was significantly greater in HIT-positive subjects (6 vs. 4, p<0.0001), though 21 (57%) HIT-negative subjects had an intermediate or high probability (≧4) score, highlighting the limited specificity of clinical diagnosis. More patients in the HIT-positive group were on the cardiovascular surgical service (66.7% vs. 37.8%) and received treatment for HIT (90.5% vs. 67.6%), though these differences did not meet statistical significance (p=0.06 for both comparisons). Other demographic and clinical features were similar between the two groups (Table 1).

TABLE 1
Demographic and clinical characteristics of
HIT-positive and HIT-negative subjects.

| | HIT-positive | HIT-negative | |
|---|---|---|---|
| Characteristic | (n = 21) | (n = 37) | p-value |
| Age: mean (range) | 66 (39-89) | 63 (39-80) | 0.47 |
| Female gender: n (%) | 9 (42.9) | 19 (51.4) | 0.59 |
| Race: n (%) | | | 0.39 |
| White | 16 (76.2) | 23 (62.2) | |
| Black | 1 (4.8) | 6 (16.2) | |
| Other/Unknown | 4 (19.0) | 8 (21.6) | |
| Patient population: n (%) | | | |
| Cardiovascular surgery | 14 (66.7) | 14 (37.8) | 0.06 |
| Surgery (non-cardiovascular) | 5 (23.8) | 9 (24.3) | |
| Medicine | 2 (9.5) | 13 (35.1) | |
| Other | 0 | 1 (2.7) | |
| Setting: n (%) | | | 0.22 |
| Tertiary care hospital | 18 (85.7) | 26 (70.3) | |
| Community hospital | 3 (14.2) | 11 (29.7) | |
| Platelet count × 10⁹/L: | 50 (19-139) | 69 (3-883) | 0.29 |
| median (range)* | | | |
| Recognized thrombosis: | 14 (66.7) | 20 (54.1) | 0.41 |
| n (%)* | | | |
| 4Ts score: median | 6 (5-7) | 4 (3-5) | <0.0001 |
| (interquartile range) | | | |
| Received treatment for | 19 (90.5) | 25 (67.6) | 0.06 |
| HIT: n (%) | | | |

*At time of HIT laboratory testing

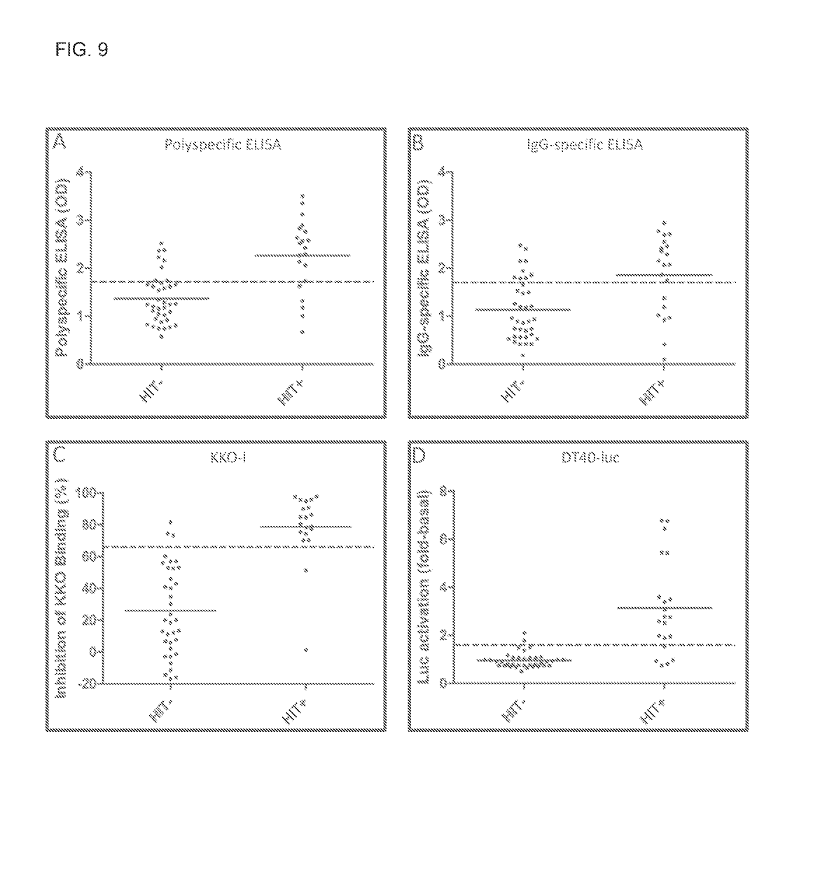

All 58 patient samples were tested using the polyspecific ELISA, IgG-specific ELISA, and DT40-luc. One HIT-positive and one HIT-negative sample were not tested by KKO-I due to insufficient sample volume. The ability of each assay to discriminate HIT-positive from HIT-negative subjects is shown in FIG. 9 (panels A-D, respectively). Consistent with previous studies, the mean OD was higher among HIT-positive than HIT-negative subject samples by both polyspecific (2.26 vs. 1.37, p<0.0001) and IgG-specific ELISA (1.86 vs. 1.14, p=0.0004). However, significant overlap in OD values among HIT-positive and HIT-negative subjects was observed with both assays (FIG. 9A-B), underscoring their limited capacity to discriminate cell-activating (and presumably pathogenic) from non-activating PF4/heparin antibodies at the level of the individual patient.

KKO-I and DT40-luc showed better diagnostic discrimination than the commercially available ELISAs (FIG. 9C-D). HIT-positive plasma samples exhibited significantly greater mean inhibition of KKO binding (78.9% vs. 26.0%, p<0.0001) (FIG. 9C) and induced significantly greater luciferase activity (3.14-fold basal vs. 0.96-fold basal, p<0.0001) than HIT-negative samples (FIG. 9D).

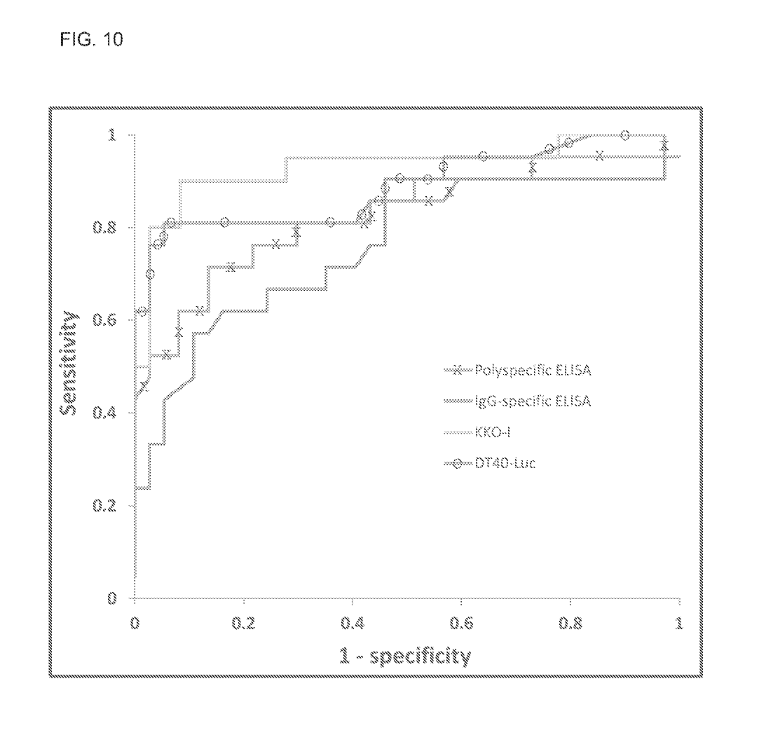

ROC curves for each assay are shown in FIG. 10. The AUC for KKO-I (0.93, 95% CI 0.85-1.00) was significantly greater than the AUC for the polyspecific (0.82, 0.70-0.95; p=0.020) and IgG-specific (0.76, 0.62-0.90; p=0.0044) ELISAs, but not the DT40-luc (0.89, 0.79-0.99; p=0.40). The AUC for DT40-luc was significantly greater than the AUC for the IgG-specific (p=0.046), but not the polyspecific ELISA (p=0.28).

Table 2 shows the sensitivity/specificity pairs at the most northwest point on the ROC curve (the point at which sensitivity and specificity are optimized) for each assay. The cut-offs associated with these points are denoted by dashed horizontal lines in FIG. 9. At a cut-off of 66%, the sensitivity and specificity of KKO-I was 0.90 and 0.92, respectively, and correctly classified 91% (51/56) of samples with respect to the reference standard. A lower cut-off of 50% improved sensitivity (0.95) at the cost of reduced specificity (0.72). The sensitivity and specificity of DT40-luc at a cut-off of 1.6-fold basal was 0.81 and 0.95, respectively, and was associated with correct classification of 88% (51/58) of samples. The operating characteristics of KKO-I and DT40-luc compared favorably with those of the polyspecific and IgG-specific ELISA. At cut-offs of 1.72 OD and 1.70 OD, the sensitivity of the latter assays was 0.76 and 0.67 and the specificity 0.78 and 0.76, respectively (Table 2). At these optimized cut-offs, the polyspecific ELISA correctly classified 78% (45/58) and the IgG-specific ELISA 72% (42/58) of samples.

TABLE 2
Operating characteristics of the assays at the most northwest
point on the receiver-operating characteristic curve.

| | | Sensitivity | Specificity |
|---|---|---|---|
| Assay | Cut-off | (95% CI) | (95% CI) |
| Polyspecific | 1.72 OD | 0.76 (0.53-0.92) | 0.78 (0.62-0.90) |
| ELISA | | | |
| IgG-specific | 1.70 OD | 0.67 (0.43-0.85) | 0.76 (0.59-0.88) |
| ELISA | | | |
| KKO-I | 66% | 0.90 (0.68-0.99) | 0.92 (0.78-0.98) |
| DT40-luc | 1.6-fold basal | 0.81 (0.58-0.95) | 0.95 (0.82-0.99) |
| Combinatorial | KKO-I ≧ 75% | 0.95 (0.74-1.00) | 0.94 (0.80-0.99) |
| | or DT40-luc ≧ | | |
| | 2-fold basal | | |

Combinatorial analysis showed that the performance of KKO-I and DT40-luc could be further improved when both assays were integrated within a single diagnostic algorithm. A combinatorial strategy in which a sample was considered positive if it demonstrated either KKO-I≧75% or DT40-luc≧2.0-fold basal would have resulted in correct classification of 55 of the 58 patients and a sensitivity and specificity of 0.95 and 0.94, respectively (Table 2). Conjunctive combinatorial strategies (i.e. Boolean operations using “AND” rather than “OR”) did not result in improved performance.

Because the polyspecific PF4/heparin ELISA has very high negative predictive value for HIT, we chose to confine our study cohort to the more diagnostically challenging patients who test positive by polyspecific ELISA (OD≧0.4). Nevertheless, samples from 9 additional subjects with suspected HIT that tested negative in the polyspecific ELISA were analyzed as negative controls. As expected, all 9 samples showed negligible inhibition of KKO binding (range: −16.1% to 11.9%) and luciferase activity (0.75-fold basal to 1.37-fold basal) (data not shown).

### Example 17

### Discussion

Binding of KKO to immobilized PF4/heparin is inhibited to a greater extent by PRP-SRA-positive plasma than by plasma from patients with non-cell-activating anti-PF4/heparin antibodies. The KKO-I assay was designed to leverage this property of KKO, which possesses in vitro and in vivo platelet-activating activity, for the purpose of discriminating cell-activating and potentially pathogenic HIT antibodies from their non-pathogenic counterparts. In the present study, plasma from HIT-positive subjects demonstrated significantly greater mean inhibition of KKO binding than HIT-negative plasma (78.9% vs. 26.0%, p<0.0001) by KKO-I (FIG. 9C). These findings suggest that human cell-activating antibodies in HIT plasma bind to epitopes on PF4 that overlap to a greater extent with KKO's binding site(s) than epitopes recognized by non-platelet activating antibodies, such as the isotype-matched monoclonal anti-PF4 antibody RTO, and point to epitope specificity as a potential major determinant of anti-PF4/heparin antibody pathogenicity.

KKO-I is not the only diagnostic assay for HIT to make use of KKO. HemosIL HIT-Ab_{(PF4-H)} (Instrumentation Laboratory, Bedford, Mass.) is a latex particle enhanced immunoturbidimetric assay in which agglutination of KKO-coated latex beads to PF4/polyvinylsulfonate complexes in solution is inhibited in the presence of human anti-PF4/heparin antibodies. Like the polyspecific PF4/heparin ELISA, HemosIL HIT-Ab(PF4-H) has high sensitivity but appears to be limited in its capacity to discriminate cell-activating from non-pathogenic antibodies. The assay was studied in a cohort of 102 subjects with suspected HIT, in which HIT was defined as an intermediate or high clinical suspicion coupled with a positive aggregometry-based functional assay. All 17 samples meeting this definition tested positive by HemosIL HIT-Ab_{(PF4-II)} (sensitivity 100%), but 16 additional HIT-negative samples also tested positive (specificity 81%).11 Although KKO-I and HemosIL HIT-Ab_{(PF4-H)} have not been compared head-to-head, our data suggest that the former may be more specific for platelet-activating antibodies. The reasons for this apparent discrepancy in specificity are unknown, but may relate to differences in the geometry and kinetics of KKO. The spatial orientation and intermolecular organization of KKO on the latex bead may permit steric inhibition by a higher proportion of anti-PF4/heparin antibodies of diverse epitope specificities compared with more stringent requirements to compete with the same antibody in solution. In addition, the epitope(s) recognized by KKO on complexes of PF4/polyvinylsulfonate in solution may differ sufficiently from those on immobilized complexes of PF4/heparin that nonpathogenic antibodies can compete effectively. Better understanding of these differences may lead to more detailed characterization of the pathogenic epitopes in HIT, which in turn, could be used to further improve assay specificity.

Previous work has shown that FcγRIIa is required for the platelet activation central to the pathogenesis of HIT. To obviate the need for fresh, reactive, donor platelets, we developed a DT40 (chicken B lymphocyte) cell line transfected with human FcγRIIa coupled to a luciferase reporter to measure cellular activation by HIT antibodies. In this platelet-free system (DT40-luc), plasma from HIT-positive subjects induced significantly greater luciferase activity than plasma from HIT-negative subjects (3.14-fold basal vs. 0.96-fold basal, p<0.0001) (FIG. 9D). Thus, this assay recapitulates the salient requirements to induce HIT, namely PF4-heparin-IgG complexes capable of activating cells via FcγRIIa, without the need for platelet-specific antigens. This is an important feature in light of emerging evidence that activation of other cell types (e.g. endothelial cells, monocytes) may contribute to the prothrombotic phenotype of HIT through this and other Fcγ receptors.

In the current study, KKO-I and DT40-luc exhibited better discrimination than a commercially available polyspecific and IgG-specific ELISA (FIG. 9). At the most northwest point on their respective ROC curves (FIG. 10), the sensitivity and specificity of the novel assays were superior to those of the commercial tests (Table 2). The sensitivity of the polyspecific and IgG-specific ELISA is lower than that reported in other studies (0.95-1.00), primarily because we used the most northwest point on the ROC curve in our analysis rather than the manufacturer-recommended cut-off of 0.40. The specificity of all four assays would likely have been enhanced had samples from patients testing negative by polyspecific PF4/heparin ELISA, a group that typically comprises 70% to 90% of a reference laboratory's test population, been included in the study cohort.

Our findings have potentially important clinical implications. The diagnosis of HIT is a high stakes enterprise. Delays in diagnosis and institution of appropriate therapy are associated with an initial 6.1% daily risk of thromboembolism, amputation, and death. Misdiagnosis, conversely, may result in unnecessary exposure of thrombocytopenic patients without HIT to direct thrombin inhibitors and their attendant 1% daily risk of major hemorrhage. The latter is a particularly prevalent problem in current practice, as highlighted by the observation that two-thirds of the 37 HIT-negative subjects in our study nonetheless received treatment for HIT (Table 1) with potential bleeding complications and unlikely benefit.

The limited specificity of currently available immunoassays and the limited availability of more specific functional assays contribute to the problem of overdiagnosis and unnecessary treatment Immunoassays such as the polyspecific PF4/heparin ELISA are simple to perform and widely used, but are unable to discriminate cell-activating and potentially pathogenic from non-pathogenic antibodies (FIG. 9A). Depending on the patient population, only 28% to 59% of samples testing positive by polyspecific ELISA also test positive by a more specific functional assay. The OD of a positive ELISA is a helpful, but relatively crude predictor of pathogenicity (FIGS. 9A-B). Modifications of the PF4/heparin ELISA that detect only antibodies of the IgG class (e.g. IgG-specific ELISA) or antibodies inhibited by excess heparin (e.g. high-dose heparin confirmatory test) enhance specificity, but false-positive results remain common with these approaches and reductions in sensitivity have also been reported.

KKO-I and DT40-luc demonstrated better diagnostic discrimination than a commercially available polyspecific and IgG-specific ELISA in the present study (FIG. 10, Table 2) and hold promise for improving the specificity of laboratory diagnosis and curtailing the current trend of overdiagnosis. Of the 37 HIT-negative subjects in our study, all would have been misdiagnosed with (and potentially treated for) HIT based on the manufacturer-recommended polyspecific ELISA OD cut-off of 0.40. Even use of an optimized cut-off corresponding to the most northwest point on the ROC curve would have resulted in misclassification of 13 of 58 (22%) subjects by polyspecific ELISA and 16 of 58 (28%) by IgG-specific ELISA. In contrast, KKO-I and DT40-luc correctly classified 33 of 36 (92%) and 35 of 37 (95%) HIT-negative patients, respectively, suggesting the potential for these novel assays to curb the current problem of over-diagnosis and unnecessary treatment.

Functional assays for HIT such as the SRA and the heparin-induced platelet activation (HIPA) test are sensitive and more specific than commercial immunoassays, but are impracticable for most clinical laboratories due to the need for fresh reactive donor platelets, radioisotope (for the SRA), and meticulous technique and platelet aggregometry (for the HIPA). KKO-I and DT40-luc may represent more feasible options for laboratories seeking to improve the specificity of the testing they offer. As a modification of the PF4/heparin ELISA, KKO-I could be relatively easily adopted by laboratories that currently perform the former assay. DT40-luc is a test of cellular activation in which donor platelets are replaced by a cell-line that can be stored at −80° C. and retrieved as needed for testing. The endpoint for cellular activation is luciferase activity, which can be measured by a standard spectrophotometer without need for radioactivity or platelet aggregometry.

Several limitations of our study deserve mention. First, the study population was relatively small and was recruited from two hospitals within a single health system. Validation in a larger multicenter study is required. Second, the PRP-SRA employed in the current study differs from the more widely used washed platelet SRA. PRP-based aggregometry assays may be less specific than washed platelet functional assays, though SRAs utilizing PRP and washed platelets have not been directly compared. Third, there is no universally accepted gold standard for HIT. In a recent meta-analysis, a 4Ts score≧4 was associated with a positive predictive value of only 22%. Positive results by SRA in the absence of clinical HIT may also occur, particularly after cardiac surgery. To overcome these limitations in the positive predictive value of the 4Ts and SRA, we used a reference standard (4Ts score≧4 and positive PRP-SRA) incorporating both clinical and laboratory criteria, as recommended by an international expert consensus panel. Nevertheless, misclassification of some subjects in our study cannot be excluded.

With this caveat in mind, two subjects that met the prespecified definition of HIT tested negative by KKO-I at a cut-off of 66% (FIG. 9C). One was a 43-year-old woman who developed thrombocytopenia, lower extremity deep vein thrombosis, and pulmonary embolism following resection of locally advanced ovarian cancer. Her 4Ts score was 7 (high probability) and her platelet count normalized rapidly on argatroban. Her plasma showed only 1% inhibition by KKO-I and 0.81-fold basal activation by DT40-luc. Interestingly, weakly positive results were obtained by polyspecific ELISA, IgG-specific ELISA, and PRP-SRA (0.67, 0.41, and 43%, respectively). Values in this range have been reported to occur in the absence of clinical HIT, 10,36 suggesting that the patient may have been misclassified by the reference standard rather than by KKO-I. The second patient was a 72-year-old man who developed thrombocytopenia, lower extremity deep vein thrombosis, and necrotic skin lesions at heparin injection sites following percutaneous repair of a celiac artery aneurysm (4Ts score=7). Thrombocytopenia resolved with cessation of heparin and initiation of argatroban. Results of the polyspecific ELISA, IgGspecific ELISA, and PRP-SRA were 1.32, 0.97, and 93%, respectively. Although the patient tested negative by KKO-I (51% inhibition), his plasma induced strong luciferase activity (6.45-fold basal) by the DT40-luc assay. This laboratory profile suggests the possibility of an uncommon variant of HIT in which cell-activating antibodies recognize complexes of heparin and cationic proteins other than PF4 such as interleukin-8 or neutrophil-activating peptide-2. Such antibodies might be detectable using functional assays (e.g. DT40-luc), but not by PF4/heparin immunoassays (e.g. KKO-I).

As suggested by this second case in which discrepant results were obtained by KKO-I and DT40-luc, a combinatorial approach may yield better performance than use of either assay alone. Indeed, an integrated algorithm in which a sample was considered positive if it demonstrated either KKO-I≧75% or DT40-luc≧2.0-fold basal would have resulted in correct classification of 55 of the 58 patients in our study and a sensitivity and specificity of 0.95 (0.74-1.00) and 0.94 (0.80-0.99), respectively (Table 2). The operating characteristics of such an approach require validation in an independent test population.

In summary, we describe a novel immunoassay (KKO-I) and a novel functional assay (DT40-luc) for the diagnosis of HIT. KKO-I and DT40-luc discriminated cell-activating and potentially pathogenic antibodies from non-activating antibodies more effectively than two commercially available ELISAs in a small cohort of patients with suspected HIT. These assays are simple to perform; do not require donor platelets, radioactivity, or platelet aggregometry; and hold promise for improving the specificity and feasibility of HIT laboratory testing. Validation of KKO-I and DT40-luc in a larger prospective study is underway.

### VI. ALTERNATE EMBODIMENTS

In one aspect is provided, a method for diagnosing heparin-induced thrombocytopenia (HIT) in a subject, the method comprising: (a) contacting a PF4-heparin complex with a biological sample from the subject; (b) contacting the composition of part (a) with an HIT-like antibody; (c) measuring the amount of bound HIT-like antibody; wherein a significant decrease in the amount of bound HIT-like antibody in the composition of part (b) as compared to a control level is indicative of a diagnosis of HIT in the subject. In another aspect, the PF4-heparin complex is bound to a surface. In another aspect, the surface is a solid support surface. In another aspect, the surface is a microsphere in solution. In another aspect, the PF4-heparin complex is in solution. In another aspect, the HIT-like antibody is the KKO antibody. In another aspect, the control level is the level of binding in the absence of a biological sample. In another aspect, the control level is the level of binding in the presence of non-platelet activating anti-PF4-heparin antibodies. In another aspect, the measuring step comprises contacting the composition of part (b) with a ligand that binds the HIT-like antibody. In another aspect, the ligand is a second antibody. In another aspect, the second antibody is an IgG antibody. In another aspect, the ligand that binds HIT-like antibodies is labeled with a reporter molecule. In another aspect, the reporter molecule is an enzyme capable of being detected by color change when contacted with a chromogenic substrate. In another aspect, the method comprises contacting the composition with the chromogenic substrate and detecting the color change via spectrophotometer. In another aspect, the enzyme is horseradish peroxidase (HRP). In another aspect, the method further comprises the step of washing the surface after contacting with the HIT-like antibody. In another aspect, the method further comprises performing one or more additional diagnostic tests to confirm the diagnosis of HIT. In another aspect, the additional diagnostic tests are one or more of a polyspecific ELISA, an IgG-specific ELISA, a cell based assay and a serotonin release assay (SRA). In another aspect, the sample is whole blood, serum, plasma or purified immunoglobulin.

In another aspect, is provided method for diagnosing the presence of platelet-activating anti-PF4-heparin antibodies in a subject, the method comprising: (a) contacting a PF4-heparin complex with a biological sample from the subject; (b) contacting the composition of part (a) with an HIT-like antibody; (c) measuring the amount of bound HIT-like antibody; wherein a significant decrease in the level of binding of the HIT-like antibody as compared to a control level indicates the presence of platelet-activating anti-PF4-heparin antibodies in the sample. In another aspect, the PF4-heparin complex is bound to a surface. In another aspect, the surface is a solid support surface. In another aspect, the surface is a microsphere in solution. In another aspect, the HIT-like antibody is the KKO antibody. In another aspect, the control level is the level of binding in the absence of a biological sample. In another aspect, the control level is the level of binding in the presence of non-platelet activating anti-PF4-heparin antibodies. In another aspect, the measuring step comprises contacting the composition of part (b) with a ligand that binds the HIT-like antibody. In another aspect, the ligand is a second antibody. In another aspect, the second antibody is an IgG antibody. In another aspect, the ligand that binds anti-HIT antibodies is labeled with a reporter molecule. In another aspect, the reporter molecule is an enzyme capable of being detected by color change when contacted with a chromogenic substrate. In another aspect the method further comprises contacting the composition with the chromogenic substrate and detecting the color change via spectrophotometer. In another aspect, the enzyme is horseradish peroxidase (HRP). In another aspect, the method further comprises the step of washing the surface after contacting with the HIT-like antibody. In another aspect, the method further comprises performing one or more diagnostic tests to confirm the diagnosis of HIT. In another aspect, the additional diagnostic tests are one or more of polyspecific ELISA, IgG-specific ELISA, a cell based assay and a serotonin release assay (SRA). In another aspect, the sample is whole blood, serum, plasma or purified immunoglobulin. In another aspect, the subject is a mammalian subject. In another aspect, the subject is a human. In another aspect, about 50% inhibition of KKO binding indicates a diagnosis of HIT in the subject or presence of platelet-activating antibodies in the sample. In another aspect, about 58% inhibition of KKO binding indicates a diagnosis of HIT in the subject or presence of platelet-activating antibodies in the sample. In another aspect, about 66% inhibition of KKO binding indicates a diagnosis of HIT in the subject or presence of platelet-activating antibodies in the sample. In another aspect, about 75% inhibition of KKO binding indicates a diagnosis of HIT in the subject or presence of platelet-activating antibodies in the sample.

In another aspect is provided an assay kit comprising one or more of: (a) PF4 bound to heparin or a heparin-like molecule; (b) a suitable aliquot of HIT-like antibody; (b) a suitable aliquot of a ligand that binds HIT-like antibodies conjugated to a reporter; (d) a suitable aliquot of a substrate which allows identification and quantification of the reporter; (e) a solid support or bead to which the PF4-heparin can bind; and (f) washing buffers.

In another aspect, is provided a method for supporting a diagnosis of heparin-induced thrombocytopenia (HIT) in a subject, the method comprising: (a) contacting a PF4-heparin complex with a biological sample from the subject; (b) contacting the composition resulting from part (a) with an HIT-like antibody; and (c) measuring the amount of bound HIT-like antibody; wherein a significant decrease in the amount of bound HIT-like antibody in the composition of part (b) as compared to a negative control or reference level, in combination with one or more positive clinical factors, is indicative of a diagnosis of HIT in the subject.

In another aspect, is provided a method for affirming a diagnosis of heparin-induced thrombocytopenia (HIT) in a subject, the method comprising: (a) contacting a PF4-heparin complex with a biological sample from the subject; (b) contacting the composition resulting from part (a) with an HIT-like antibody; and (c) measuring the amount of bound HIT-like antibody, wherein a significant decrease in the amount of bound HIT-like antibody in the composition of part (b) as compared to a negative control or reference level, in combination with one or more positive clinical factors, is indicative of a diagnosis of HIT in the subject.

In another aspect, is provided a method for diagnosing an increased likelihood of heparin-induced thrombocytopenia (HIT) in a subject, the method comprising: (a) contacting a PF4-heparin complex with a biological sample from the subject; (b) contacting the composition resulting from part (a) with an HIT-like antibody; and (c) measuring the amount of bound HIT-like antibody, wherein a significant decrease in the amount of bound HIT-like antibody in the composition of part (b) as compared to a negative control or reference level, in combination with one or more positive clinical factors, is indicative of an increased probability that the subject has HIT.

In another aspect, is provided a method for helping to rule out a diagnosis of HIT in a subject, the method comprising: (a) contacting a PF4-heparin complex with a biological sample from the subject; (b) contacting the composition resulting from part (a) with an HIT-like antibody; and (c) measuring the amount of bound HIT-like antibody, wherein a significant increase in the amount of bound HIT-like antibody in the composition of part (b) as compared to a positive control or reference level indicates that the subject is less likely to have HIT.

In another aspect, is provided a method for ruling out a diagnosis of HIT in a subject, the method comprising: (a) contacting a PF4-heparin complex with a biological sample from the subject; (b) contacting the composition resulting from part (a) with an HIT-like antibody; and (c) measuring the amount of bound HIT-like antibody, wherein a significant increase in the amount of bound HIT-like antibody in the composition of part (b) as compared to a positive control or eference level, in combination with other negative clinical factors, indicates that the subject does not have HIT.

In another aspect, is provided a method for helping to rule out a diagnosis of HIT in a subject, the method comprising: (a) contacting a PF4-heparin complex with a biological sample from the subject; (b) contacting the composition resulting from part (a) with an HIT-like antibody; and (c) measuring the amount of bound HIT-like antibody, wherein a significant increase in the amount of bound HIT-like antibody in the composition of part (b) as compared to a positive control or reference level, in combination with other negative clinical factors, indicates that the subject is less likely to have HIT.

In another aspect, the PF4-heparin complex is bound to a surface. In another aspect, the surface is a solid support surface. In another aspect, the surface is a microsphere in solution. In another aspect, the HIT-like antibody is the KKO antibody. In another aspect, the negative control level is the level of binding in the absence of a biological sample or in the presence of non-platelet activating anti-PF4-heparin antibodies. In another aspect, the positive control level is the level of binding in the presence of platelet-activating antibodies. In another aspect, the measuring step comprises contacting the composition of part (b) with a ligand that binds the HIT-like antibody. In another aspect, the ligand is a second antibody. In another aspect, the second antibody is an IgG antibody. In another aspect, the ligand that binds anti-HIT antibodies is labeled with a reporter molecule. In another aspect, the reporter molecule is an enzyme capable of being detected by color change when contacted with a chromogenic substrate. In another aspect, the method comprises contacting the composition with the chromogenic substrate and detecting the color change via spectrophotometer. In another aspect, the enzyme is horseradish peroxidase (HRP). In another aspect, the method comprises the step of washing the surface after contacting with the HIT-like antibody. In another aspect, the method further comprises performing one or more diagnostic tests to confirm the diagnosis of HIT. In another aspect, the additional diagnostic tests are one or more of polyspecific ELISA, IgG-specific ELISA, a cell based assay and a serotonin release assay (SRA). In another aspect, the sample is whole blood, serum, plasma or purified immunoglobulin. In another aspect, the subject is a mammalian subject. In another aspect, the subject is a human. In another aspect, the positive clinical factors are one or more of an intermediate or high probability of HIT as defined by 4Ts score, development of one or more thromboembolic complications (TEC), trauma/orthopedic surgery, thrombocytopenia, enlargement or extension of a previously diagnosed blood clot, development of a new blood clot, stroke, myocardial infarction, acute leg ischemia, deep vein thrombosis (DVT), pulmonary embolism (PE); systemic reaction beginning at the site of heparin infusion, including fever, chills, high blood pressure, a fast heart rate, shortness of breath, chest pain, and rash.

All publications and priority patent applications, U.S. Provisional Patent Application Nos. 61/640,960 and 61/790,953, cited in this specification are incorporated herein by reference. While the invention has been described with reference to particular embodiments, it will be appreciated that modifications can be made without departing from the spirit of the invention. Such modifications are intended to fall within the scope of the appended claims.

### REFERENCES

1. Althaus K, Strobel U, Warkentin T E, Greinacher A. Combined use of the high heparin step and optical density to optimize diagnostic sensitivity and specificity of an anti-PF4/heparin enzyme-immunoassay. Thromb Res. 2011; 128(3):256-260.

2. Amiral J, Bridey F, Dreyfus M, et al. Platelet factor 4 complexed to heparin is the target for antibodies generated in heparin-induced thrombocytopenia. Thrombosis and Haemostasis. 1992; 68:95-96.

3. Amiral J, Marfaing-Koka A, Wolf M, et al. Presence of autoantibodies to interleukin-8 or neutrophil-activating peptide-2 in patients with heparin-associated thrombocytopenia. Blood. 1996; 88(2):410-416.

4. Arepally G M, Kamei S, Park K S, et al. Characterization of a murine monoclonal antibody that mimics heparin-induced thrombocytopenia antibodies. Blood. 2000; 95(5):1533-1540.

5. Arepally G M, Ortel T L. Clinical practice. Heparin-induced thrombocytopenia. N Engl J Med. 2006; 355(8):809-817.

6. Bakchoul T, Giptner A, Najaoui A, Bein G, Santoso S, Sachs U J. Prospective evaluation of PF4/heparin immunoassays for the diagnosis of heparin-induced thrombocytopenia. J Thromb Haemost. 2009; 7(8):1260-1265.

7. Bauer T L, Arepally G, Konkle B A, et al. Prevalence of heparin-associated antibodies without thrombosis in patients undergoing cardiopulmonary bypass surgery. Circulation. 1997; 95(5):1242-1246.

8. Blank M, Schoenfeld Y, Tavor S, et al. Anti-platelet factor 4/heparin antibodies from patients with heparin-induced thrombocytopenia provoke direct activation of microvascular endothelial cells. Int Immunol. 2002; 14(2):121-129.

9. Bock P E, Luscombe M, Marshall S E, Pepper D S, Holbrook J J. The multiple complexes formed by the interaction of platelet factor 4 with heparin. Biochemical Journal. 1980; 191:769-776.

10. Chong B H, Fawaz I, Chesterman C N, Berndt M C. Heparin-induced thrombocytopenia: mechanism of interaction of the heparin-dependent antibody with platelets. Br J Haematol. 1989; 73(2):235-240.

11. Cines D B, Kaywin P, Bina M, Tomaski A, Schreiber A D. Heparin-associated thrombocytopenia. N Engl J Med. 1980; 303(14):788-795.

12. Crowther M A, Cook D J, Albert M, et al. The 4Ts scoring system for heparin-induced thrombocytopenia in medical-surgical intensive care unit patients. J Crit Care. 2010; 25(2):287-293. Prepublished on 2010 Feb. 13 as DOI S0883-9441(10)00006-7 [pii]

13. Cuker A, Cines D B. How I treat heparin-induced thrombocytopenia. Blood. 2012; 119(10):2209-2218.

14. Cuker A, Gimotty P A, Crowther M A, Warkentin T E. Predictive value of the 4Ts scoring system for heparin-induced thrombocytopenia: a systematic review and meta-analysis. Blood. 2012. In press.

15. Cuker A, Ortel T L. ASH evidence-based guidelines: is the IgG-specific anti-PF4/heparin ELISA superior to the polyspecific ELISA in the laboratory diagnosis of HIT? Hematology Am Soc Hematol Educ Program. 2009; 250-252.

16. Cuker A. Heparin-induced thrombocytopenia (HIT) in 2011: an epidemic of overdiagnosis. Thromb Haemost. 2011; 106(6):993-994.

17. DeLong E R, DeLong D M, Clarke-Pearson D L. Comparing the areas under two or more correlated receiver operating characteristic curves: a nonparametric approach. Biometrics. 1988; 44(3):837-845.

18. Demma L J, Winkler A M, Levy J H. A diagnosis of heparin-induced thrombocytopenia with combined clinical and laboratory methods in cardiothoracic surgical intensive care unit patients. Anesth Analg. 2011; 113(4):697-702. Prepublished on 2011 Jul. 27 as DOI 10.1213/ANE.0b013e3182297031.

19. Denys B, Stove V, Philippe J, Devreese K. A clinical-laboratory approach contributing to a rapid and reliable diagnosis of heparin-induced thrombocytopenia. Thromb Res. 2008; 123(1):137-145. Prepublished on 2008 Jun. 28 as DOI 10.1016/j.thromres.2008.04.020.

20. Evans E, Ritchie K. Dynamic strength of molecular adhesion bonds. Biophys J. 1997; 72(4):1541-1555. Prepublished on 1997 Apr. 1 as DOI 10.1016/50006-3495(97)78802-7.

21. Greinacher A, Eichler P, Lubenow N, Kwasny H, Luz M. Heparin-induced thrombocytopenia with thromboembolic complications: meta-analysis of 2 prospective trials to assess the value of parenteral treatment with lepirudin and its therapeutic aPTT range. Blood. 2000; 96(3):846-851.

22. Greinacher A, Gopinadhan M, Gunther J U, et al. Close approximation of two platelet factor 4 tetramers by charge neutralization forms the antigens recognized by HIT antibodies. Arterioscler Thromb Vasc Biol. 2006; 26(10):2386-2393.

23. Greinacher A, Ittermann T, Bagemuhl J, et al. Heparin-induced thrombocytopenia: towards standardization of platelet factor 4/heparin antigen tests. Journal of thrombosis and haemostasis: JTH. 2010;8(9):2025-2031. Prepublished on 2010 Jul. 16 as DOI 10.1111/j.1538-7836.2010.03974.x.

24. Greinacher A, Michels I, Kiefel V, Mueller-Eckhardt C. A rapid and sensitive test for diagnosing heparin-associated thrombocytopenia. Thromb Haemost. 1991; 66(6):734-736.

25. Greinacher A, Potzsch B, Amiral J, Dummel V, Eichner A, Mueller-Eckhardt C. Heparin-associated thrombocytopenia: isolation of the antibody and characterization of a multimolecular PF4-heparin complex as the major antigen. Thromb Haemost. 1994; 71(2):247-251.

26. Janatpour K A, Gosselin R C, Dager W E, et al. Usefulness of optical density values from heparin-platelet factor 4 antibody testing and probability scoring models to diagnose heparin-induced thrombocytopenia. Am J Clin Pathol. 2007; 127(3):429-433.

27. Juhl D, Eichler P, Lubenow N, Strobel U, Wessel A, Greinacher A. Incidence and clinical significance of anti-PF4/heparin antibodies of the IgG, IgM, and IgA class in 755 consecutive patient samples referred for diagnostic testing for heparin-induced thrombocytopenia. Eur J Haematol. 2006; 76(5):420-426.

28. Kasthuri R S, Glover S L, Jonas W, et al. PF4/heparin-antibody complex induced monocyte tissue factor expression and release of tissue factor positive microparticles by activation of FcγRI. Blood. 2012; 119(22):5285-5293.

29. Kelton J G, Sheridan D, Santos A, et al. Heparin-induced thrombocytopenia: laboratory studies. Blood. 1988; 72:925-930.

30. Kelton J G, Smth J W, Warkentin T E, Hayward C P, Denomme G A, Horsewood P. Immunoglobulin G from patients with heparin-induced thrombocytopenia binds to a complex of heparin and platelet factor 4. Blood. 1994; 83(11):3232-3239.

31. Legnani C, Cini M, Pili C, Boggian O, Frascaro M, Palareti G. Evaluation of a new automated panel of assays for the detection of anti-PF4/heparin antibodies in patients suspected of having heparin-induced thrombocytopenia. Thromb Haemost. 2010; 104(2):402-409.

32. Lewis B E, Wallis D E, Hursting M J, Levine R L, Leya F. Effects of argatroban therapy, demographic variables, and platelet count on thrombotic risks in heparin-induced thrombocytopenia. Chest. 2006; 129(6):1407-1416.

33. Li Z Q, Liu W, Park K S, et al. Defining a second epitope for heparin-induced thrombocytopenia/thrombosis (HIT/T) antibodies using KKO, a murine HIT-like monoclonal antibody. Blood. 2003; 99:1230-1236.

34. Litvinov R I, Barsegov V, Schissler A J, et al. Dissociation of bimolecular alphaIIbbeta3-fibrinogen complex under a constant tensile force. Biophys J. 2011; 100(1):165-173. Prepublished on 2010 Dec. 31 as DOI 10.1016/j.bpj.2010.11.019.

35. Litvinov R I, Bennett J S, Weisel J W, Shuman H. Multi-step fibrinogen binding to the integrin (alpha)IIb(beta)3 detected using force spectroscopy. Biophys J. 2005; 89(4):2824-2834.

36. Litvinov R I, Gorkun O V, Owen S F, Shuman H, Weisel J W. Polymerization of fibrin: specificity, strength, and stability of knob-hole interactions studied at the single-molecule level. Blood. 2005; 106(9):2944-2951. Prepublished on 2005 Jul. 7 as DOI 10.1182/blood-2005-05-2039.

37. Litvinov R I, Shuman H, Bennett J S, Weisel J W. Binding strength and activation state of single fibrinogen-integrin pairs on living cells. Proc Natl Acad Sci USA. 2002; 99(11):7426-7431.

38. Litvinov R I, Yakovlev S, Tsurupa G, Gorkun O V, Medved L, Weisel J W. Direct evidence for specific interactions of the fibrinogen alphaC-domains with the central E region and with each other. Biochemistry. 2007; 46(31):9133-9142. Prepublished on 2007 Jul. 17 as DOI 10.1021/bi700944j.

39. Lo G K, Juhl D, Warkentin T E, Sigouin C S, Eichler P, Greinacher A. Evaluation of pretest clinical score (4 T's) for the diagnosis of heparin-induced thrombocytopenia in two clinical settings. J Thromb Haemost. 2006;4(4):759-765.

40. Lo G K, Sigouin C S, Warkentin T E. What is the potential for overdiagnosis of heparin-induced thrombocytopenia? Am J Hematol. 2007; 82(12):1037-1043. Prepublished on 2007 Aug. 28 as DOI 10.1002/ajh.21032.

41. Long M, Lu S, Sun G. Kinetics of receptor-ligand interactions in immune responses. Cell Mol Immunol. 2006; 3(2):79-86. Prepublished on 2006 May 16 as DOI.

42. Lubenow N, Eichler P, Lietz T, Greinacher A; Hit Investigators Group. Lepirudin in patients with heparin-induced thrombocytopenia—results of the third prospective study (HAT-3) and a combined analysis of HAT-1, HAT-2, and HAT-3. J Thromb Haemost. 2005; 3(11):2428-2436.

43. Oliveira G B, Crespo E M, Becker R C, et al. Incidence and prognostic significance of thrombocytopenia in patients treated with prolonged heparin therapy. Arch Intern Med. 2008; 168(1):94-102. Prepublished on 2008 Jan. 16 as DOI 10.1001/archinternmed.2007.65.

44. Park K S, Rifat S, Eck H, Adachi K, Surrey S, Poncz M. Biologic and biochemic properties of recombinant platelet factor 4 demonstrate identity with the native protein. Blood. 1990; 75(6):1290-1295.

45. Pouplard C, Amiral J, Borg J-Y, Laporte-Simitsidis S, Delhousee B, Gruel Y. Decision analysis for use of platelet-aggregation tests, carbon 14C-serotonin release assay, and heparin-platelet factor 4 enzyme-linked immunosorbent assay for diagnosis of heparin-induced thrombocytopenia. Am J Clin Pathol. 1999; 111:700-706.

46. Pouplard C, Gueret P, Fouassier M, Ternisien C, Trossaert M, Regina S, Gruel Y. Prospective evaluation of the ‘4Ts’ score and particle gel immunoassay specific to heparin/PF4 for the diagnosis of heparin-induced thrombocytopenia. J Thromb Haemost. 2007; 5(7):1373-1379.

47. Rauova L, Hirsch J D, Greene T K, et al. Monocyte-bound PF4 in the pathogenesis of heparin-induced thrombocytopenia. Blood. 2010; 116(23):5021-5031.

48. Rauova L, Poncz M, McKenzie S E, et al. Ultralarge complexes of PF4 and heparin are central to the pathogenesis of heparin-induced thrombocytopenia. Blood. 2005; 105(1):131-138.

49. Rauova L, Zhai L, Kowalska M A, Arepally G M, Cines D B, Poncz M. Role of platelet surface PF4 antigenic complexes in heparin-induced thrombocytopenia pathogenesis: diagnostic and therapeutic implications. Blood. 2006; 107(6):2346-2353.

50. Reilly M P, Taylor S M, Hartman N K, et al. Heparin-induced thrombocytopenia/thrombosis in a transgenic mouse model requires human platelet factor 4 and platelet activation through FcRgIIa. Blood. 2000; 98:2442-2447.

51. Sachais B S, Litvinov R I, Yarovoi S V, et al. Dynamic antibody-binding properties in the pathogenesis of HIT. Blood. 2012; 120(5):1137-1142.

52. Sachais B S, Rux A H, Cines D B, et al. Rational design and characterization of platelet factor 4 antagonists for the study of heparin-induced thrombocytopenia. Blood. 2012; 119(25):5955-5962.

53. Schenk S, El-Banayosy A, Morshuis M, et al. IgG classification of anti-PF4/heparin antibodies to identify patients with heparin-induced thrombocytopenia during mechanical circulatory support. J Thromb Haemost. 2007; 5(2):235-241. Prepublished on 2006 Nov. 7 as DOI 10.1111/j.1538-7836.2007.02295.x.

54. Sheridan D, Carter C, Kelton J G. A diagnostic test for heparin-induced thrombocytopenia. Blood. 1986; 67(1):27-30.

55. Suvarna S, Espinasse B, Qi R, et al. Determinants of PF4/heparin immunogenicity. Blood. 2007; 110(13):4253-4260.

56. Tsurupa G, Pechik I, Litvinov R I, et al. On the mechanism of alphaC polymer formation in fibrin. Biochemistry. 2012; 51(12):2526-2538.

57. Visentin G P, Ford S E, Scott J P, Aster R H. Antibodies from patients with heparin-induced thrombocytopenia/thrombosis are specific for platelet factor 4 complexed with heparin or bound to endothelial cells. J Clin Invest. 1994; 93(1):81-88.

58. Warkentin T E, Greinacher A, Gruel Y, Aster R H, Chong B H; scientific and standardization committee of the international society on thrombosis and haemostasis. Laboratory testing for heparin-induced thrombocytopenia: a conceptual framework and implications for diagnosis. J Thromb Haemost. 2011; 9(12):2498-2500.

59. Warkentin T E, Levine M N, Hirsh J, Horsewood P, Roberts R S, Gent M, Kelton J G. Heparin-induced thrombocytopenia in patients treated with low-molecular-weight heparin or unfractionated heparin. N Engl J Med. 1995; 332(20):1330-1335.

60. Warkentin T E, Sheppard J I, Moore J C, Sigouin C S, Kelton J G. Quantitative interpretation of optical density measurements using PF4-dependent enzyme-immunoassays. J Thromb Haemost. 2008; 6(8):1304-1312. Prepublished on 2008 May 21 as DOI JTH3025 [pii]

61. Warkentin T E. Heparin-induced thrombocytopenia in the ICU: a transatlantic perspective. Chest. 2012; 142(4):815-816.

62. Warkentin T E. How I diagnose and manage HIT. Hematology American Society of Hematology Education Program. 2011:143-149.

63. Weisel J W, Shuman H, Litvinov R I. Protein-protein unbinding induced by force: single-molecule studies. Curr Opin Struct Biol. 2003; 13(2):227-235. Prepublished on 2003 May 3 as DOI S0959440X03000393 [pii].

64. Whitlatch N L, Kong D F, Metjian A D, Arepally G M, Ortel T L. Validation of the high-dose heparin confirmatory step for the diagnosis of heparin-induced thrombocytopenia. Blood. 2010; 116(10):1761-1766.

65. Ziporen L, Li Z Q, Park K S, et al. Defining an antigenic epitope on platelet factor 4 (PF4) associated with heparin-induced thrombocytopenia (HIT). Blood. 1998; 92:3250-3259.

66. Zwicker J I, Uhl L, Huang W Y, Shaz B H, Bauer K A. Thrombosis and ELISA optical density values in hospitalized patients with heparin-induced thrombocytopenia. J Thromb Haemost. 2004; 2(12):2133-2137. Prepublished on 2004 Dec. 23 as DOI 10.1111/j.1538-7836.2004.01039.x.

1. (canceled)

2. A method for diagnosing the presence of platelet-activating anti-PF4-heparin antibodies in a subject, the method comprising:
(a) contacting a PF4-heparin complex with a biological sample from the subject;
(b) contacting the composition of part (a) with an HIT-like antibody;
(c) measuring the amount of bound HIT-like antibody;
wherein a significant decrease in the level of binding of the HIT-like antibody as compared to a control level indicates the presence of platelet-activating anti-PF4-heparin antibodies in the sample.

3-5. (canceled)

6. A method for ruling out a diagnosis of HIT in a subject, the method comprising:
(a) contacting a PF4-heparin complex with a biological sample from the subject;
(b) contacting the composition resulting from part (a) with an HIT-like antibody; and
(c) measuring the amount of bound HIT-like antibody,
wherein a significant increase in the amount of bound HIT-like antibody in the composition of part (b) as compared to a positive control or reference level, in combination with other negative clinical factors, indicates that the subject does not have HIT, or is less likely to have HIT.

7-8. (canceled)

9. The method according to claim 2, wherein the PF4-heparin complex is bound to a surface or is in solution.

10. The method according to claim 9, wherein the surface is a solid support surface or a microsphere in solution.

11. The method according to claim 2, wherein the HIT-like antibody is the KKO antibody.

12. The method according to claim 2, wherein the control level is selected from the level of binding in the absence of a biological sample and the level of binding in the presence of non-platelet activating anti-PF4-heparin antibodies.

13. The method according to claim 2, wherein the measuring step comprises contacting the composition of part (b) with a ligand that binds the HIT-like antibody.

14. The method according to claim 13, wherein the ligand is a second antibody.

15. (canceled)

16. The method according to claim 13, wherein the ligand that binds HIT-like antibodies is labeled with a reporter molecule.

17. The method according to claim 16, wherein the reporter molecule is an enzyme capable of being detected by color change when contacted with a chromogenic substrate.

18. (canceled)

19. The method according to claim 17, wherein the enzyme is horseradish peroxidase (HRP).

20. The method according to claim 31, further comprising performing one or more additional diagnostic tests to confirm the diagnosis of HIT.

21. The method according to claim 20, wherein the additional diagnostic tests are one or more of a polyspecific ELISA, an IgG-specific ELISA, a cell based assay and a serotonin release assay (SRA).

22. The method according to claim 2, wherein the sample is whole blood, serum, plasma or purified immunoglobulin.

23. (canceled)

24. The method according to claim 2, wherein the subject is a human.

25. The method according to claim 11, wherein about 50% inhibition or greater of KKO binding indicates a diagnosis of HIT in the subject or presence of platelet-activating antibodies in the sample.

26-28. (canceled)

29. The method according to claim 32, wherein the positive clinical factors are one or more of an intermediate or high probability of HIT as defined by 4Ts score, development of one or more thromboembolic complications (TEC), trauma/orthopedic surgery, thrombocytopenia, enlargement or extension of a previously diagnosed blood clot, development of a new blood clot, stroke, myocardial infarction, acute leg ischemia, deep vein thrombosis (DVT), pulmonary embolism (PE); systemic reaction beginning at the site of heparin infusion, including fever, chills, high blood pressure, a fast heart rate, shortness of breath, chest pain, and rash.

30. An assay kit comprising one or more of:
(a) PF4 bound to heparin or a heparin-like molecule;
(b) a suitable aliquot of HIT-like antibody
(b) a suitable aliquot of a ligand that binds HIT-like antibodies conjugated to a reporter;
(d) a suitable aliquot of a substrate which allows identification and quantification of the reporter;
(e) a solid support or bead to which the PF4-heparin can bind; and
(f) washing buffers.

31. The method according to claim 2, wherein the presence of platelet-activating anti-PF4-heparin antibodies in the sample is indicative of:
(i) a diagnosis of diagnosing heparin-induced thrombocytopenia (HIT) in the subject; or
(ii) an increased risk of HIT in the subject.

32. The method according to claim 2, wherein the presence of platelet-activating anti-PF4-heparin antibodies in the sample in combination with one or more positive clinical factors, is indicative of:
(i) a diagnosis of diagnosing heparin-induced thrombocytopenia (HIT) in the subject; or
(ii) an increased risk of HIT in the subject.
